(19) United States
(12) Patent Application Publication
(10) Pub. No.: US 2026/0240874 A1
(43) Pub. Date: Aug. 20, 2026

(54) PRENYLATED CANNABINOIDS AND THEIR DERIVATIVES FOR THE TREATMENT OF CANCER

(71) Applicant: Abstrax Tech Inc., Tustin, CA (US)

(72) Inventors: Iain W.H. Oswald, Tustin, CA (US); Bryan Olivares, Tustin, CA (US); Thomas J. Martin, JR., Tustin, CA (US); Andrew Patron, Tustin, CA (US)

(21) Appl. No.: 19/539,920

(22) Filed: Feb. 13, 2026

Related U.S. Application Data
(60) Provisional application No. 63/758,737, filed on Feb. 14, 2025.
(60) Provisional application No. 63/789,880, filed on Apr. 16, 2025.

(51) Int. Cl. A61K 31/00; A61P 35/00; C07C 39/19

(52) U.S. Cl. CPC A61K 31/658; A61P 35/00; C07C 39/19

(57) ABSTRACT

A composition includes a prenylated cannabinoid in an amount effective to modulate cannabinoid receptor CB2 activity; and a pharmaceutically or nutraceutically acceptable carrier; wherein the prenylated cannabinoid has the following formula (I):

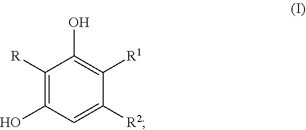

R at each occurrence is farnesyl or geranylgeranyl; R¹ at each occurrence is hydrogen or —COOR³; R² at each occurrence is —C₃-C₂₀ alkyl; and R³ at each occurrence is selected from hydrogen, —C₁-C₂₀ alkyl, and —C₁-C₂₀ haloalkyl.

### CROSS REFERENCE TO RELATED APPLICATIONS

This application claims the benefit of U.S. Provisional Application Nos. 63/758,737 filed Feb. 14, 2025, and 63/789,880 filed Apr. 16, 2025, the disclosures of which are incorporated herein by reference in its entirety.

### FIELD

The present technology relates to various compositions, products, and methods related to prenylated cannabinoids. In particular, the present technology relates to compositions products, and methods containing a novel class of prenylated cannabinoids, including sesqui-cannabigerol (SCBG), sesqui-cannabigeranylgerol (SCBGG), sesqui-cannabigerophorol (SCBGP), sesqui-cannabigerovarin (SCBGV), their corresponding acidic derivatives, and methyl ester derivatives.

### BACKGROUND

The following discussion is provided to aid the reader in understanding the disclosure and is not admitted to describe or constitute prior art thereto.

Cannabis produces a myriad of biologically active compounds, including tetrahydrocannabinol (THC), cannabidiol (CBD), and over 100 other cannabinoids. These metabolites are biosynthetically produced in their acidic forms, such as tetrahydrocannabinolic acid (THCA) and cannabidiolic acid (CBDA), which often have different properties than their neutral forms. THC, the main psychotropic compound produced by cannabis, is known for its psychoactive properties as well as its therapeutic potential, including analgesic and antiemetic effects.

CBD has also garnered much attention due to its potential health benefits without the intoxicating effects brought on by THC. It has been studied for its potential anti-inflammatory, anxiolytic, antiepileptic, and neuroprotective properties.

Other cannabinoids such as cannabigerol (CBG) have likewise seen an increase in interest due to their potential therapeutic applications. These include cannabinol (CBN), known for its potential sedative effects; cannabichromene (CBC), which may have anti-inflammatory and analgesic properties; and tetrahydrocannabivarin (THCV), which is being researched for appetite suppression and potential antidiabetic effects.

The pharmacological action of many of these cannabinoids involves cannabinoid receptors, namely, cannabinoid type-1 (CB1) and cannabinoid type-2 (CB2) receptors. For instance, THC acts as a partial agonist at the CB1 receptor, primarily found in the central nervous system, leading to its psychoactive intoxicating effects. It also interacts with the CB2 receptor, associated with the immune system, contributing to its anti-inflammatory properties. CBD, in contrast, has a low affinity for both CB1 and CB2 receptors but can act as a negative allosteric modulator of CB1, attenuating the effects of THC. Additionally, CBD interacts with other receptors such as the serotonin 5-HT1A receptor and TRPV1 receptors, contributing to its anxiolytic and analgesic effects without causing intoxication. CBG acts as a partial agonist at both CB1 and CB2 receptors but with lower efficacy compared to THC. It also interacts with a-2 adrenergic receptors and serotonin receptors, which may contribute to its potential antidepressant and analgesic effects. Unlike THC, CBG does not produce psychoactive effects, highlighting its therapeutic potential without intoxication.

More recently, a double-blind, placebo-controlled study on human subjects have found that CBG may exhibit anxiolytic properties with no intoxicating effects (Cuttler et al., Sci. Rep., 2024, 14, 16163), making it and related compounds interesting targets for anti-anxiety or stress reduction applications.

Cancer remains one of the leading causes of morbidity and mortality worldwide. Triple-negative breast cancer (TNBC), characterized by the absence of estrogen, progesterone, and HER2 receptors, is particularly aggressive and lacks targeted therapies. Recent studies have highlighted the potential of cannabinoid compounds in modulating cancer cell proliferation, apoptosis, and the tumor microenvironment. However, while compounds such as CBD and CBG have been explored, there remains a need for novel agents with enhanced selectivity and efficacy.

A novel yet underreported compound related to cannabinoids is sesqui-cannabigerol (SCBG). This compound was detected in fiber hemp in 2011 (Pollastro et al., J. Nat. Prod. 2011, 74, 9, 2019-2022).

Despite the promising therapeutic potential of SCBG, and related prenylated cannabinoids, their extremely low natural abundance in cannabis plants (<0.01% extraction yield) poses a significant barrier to their practical application in medical and consumer products. Current cannabis formulations lack sufficient concentrations of these compounds to achieve desired therapeutic effects. The extremely low natural abundance of prenylated cannabinoids in cannabis plants has limited their availability for research and commercial applications. Traditional extraction methods yield insufficient quantities for practical formulation into consumer products or pharmaceutical preparations. Furthermore, synthetic routes for producing these compounds have not been fully developed or optimized, and methods for isolating them from natural sources remain inefficient. As a result, the therapeutic potential of prenylated cannabinoids has remained largely unexplored.

Therefore, there exists a need for compositions and formulations that include prenylated cannabinoids in amounts sufficient to exert therapeutic effects, as well as methods for synthesizing and isolating these compounds. Utilizing SCBG and other prenylated cannabinoid analogs at efficacious concentrations would enable broader access to the therapeutic benefits of prenylated cannabinoids, such as e.g., conditions associated with CB2 receptor modulation, and would provide alternatives to existing cannabinoid therapies that either lack receptor selectivity or produce undesirable psychoactive effects.

### SUMMARY

The following presents a simplified summary in order to provide a basic understanding of some aspects of the disclosed subject matter. This summary is not an extensive overview, and it is not intended to identify key/critical elements or to delineate the scope thereof. Its sole purpose is to present some concepts in a simplified form as a prelude to the more detailed description that is presented later.

Various embodiments relate to novel prenylated cannabinoid compositions and methods for their use in formulations. According to a first aspect, the present technology provides methods for isolating prenylated cannabinoids in cannabis, synthetic protocols, and formulations containing sesqui-cannabigerol (SCBG), its acidic analog SCBGA, sesqui-cannabigerovarin (SCBGV), its acidic form SCBGVA, among other related compounds. These prenylated cannabinoids exhibit strong affinity for the cannabinoid CB2 receptor, suggesting significant therapeutic potential, particularly in reducing anxiety and paranoia without intoxicating effects. Further, the high CB2 receptor affinity suggests potential anti-inflammatory effects, highlighting the breadth of issues these compounds may help with.

In one aspect, the present technology provides compositions or formulations comprising effective amounts of SCBG, SCBGA, SCBGG, SCBGP, SCBGV, SCBGVA, and related prenylated cannabinoids in non-natural mixtures. For example, that the relative-% in these compositions or formulations can be near or below the % found naturally, but added to non-natural additives and mixtures, or the relative-% in these compositions or formulations can be significantly above the % found naturally. For example, these formulations may be combined with natural or synthetic carriers and excipients commonly used in food, beverages, flavors, cannabis oils, pharmaceuticals, and other consumer products to enhance bioavailability and efficacy. For compositions described herein and containing a combination of SCBG, SCBGA, SCBGG, SCBGP, SCBGV, SCBGVA, and their analogs and related prenylated cannabinoids and other ingredients is not a combination found in nature, that is, the cannabinoid or prenylated cannabinoid compound does not naturally occur with the other ingredients, or does not naturally occur in the amounts and ratios present in the combination. Thus, the formulation or composition described herein is not a naturally occurring composition or one that is found in nature.

In certain embodiments, the SCBG, SCBGA, SCBGG, SCBGP, SCBGV, SCBGVA, and their analogs and related prenylated cannabinoids may be exogenously added to the composition or endogenously present in the compositions. In certain embodiments, the SCBG, SCBGA, SCBGG, SCBGP, SCBGV, SCBGVA, and their analogs and related prenylated cannabinoids may be exogenously added to the composition.

In another aspect, the present technology provides methods for synthesizing SCBG, SCBGA, SCBGG, SCBGP, SCBGV, SCBGVA, and their analogs. The synthetic methods enable the production of these compounds in sufficient quantities for practical use in various formulations. By following modified procedures based on known synthesis routes for similar compounds, the present technology provides a novel approach to obtaining these specific prenylated cannabinoids that were not previously synthesized.

In another aspect, the present technology provides methods for isolating SCBG from cannabis itself. Specifically, the present technology provides a protocol for isolating SCBG by refining it directly from cannabis. The protocol involves extracting the oil fraction of cannabis using a suitable solvent, such as butane or ethanol, followed by refining the extract to remove volatiles, which yields a cannabinoid- and prenylated cannabinoid-rich fraction. The prenylated cannabinoids, including SCBG, are further separated from other cannabinoids using wiped film distillation. This natural isolation method enables the production of SCBG for use in various formulations, providing an approach to harnessing the potential of prenylated cannabinoids from their natural sources.

In another aspect, the present technology provides formulations that incorporate natural extracts containing grifolin, a bioactive compound known for its potential anticancer, antimicrobial, and anti-inflammatory properties. These extracts may be derived from various botanical sources such as Peperomia galioides, Rhododendron dauricum, Solanum lycopersicum (tomato), and Albatrellus ovinus (sheep polypore mushroom). By combining grifolin-containing extracts with cannabinoids and other active pharmaceutical ingredients (APIs), the present technology enhances the therapeutic potential of the formulation through synergistic effects. Grifolin's ability to modulate cellular pathways may complement the action of cannabinoids, leading to formulations with broader pharmacological profiles.

In yet another aspect, the present technology encompasses methods of using these formulations to modulate the CB2 receptor activity in mammals. Molecular docking studies have confirmed that these prenylated cannabinoids, including compounds with alkyl side chain lengths ranging from one to ten carbons, exhibit strong CB2 receptor affinity (binding energies stronger than-9 kcal/mol) and low affinity for the CB1 receptor (binding energies weaker than-7 kcal/mol). The acidic forms of these compounds also demonstrate significant receptor interactions, indicating their potential therapeutic benefits as well.

By providing formulations with elevated concentrations of prenylated cannabinoids and novel methods for their synthesis, the present technology addresses the limitations posed by the extremely low natural abundance of these compounds in cannabis plants. This advancement unlocks the full therapeutic potential of SCBG and its analogs, offering new avenues for treating cancer, anxiety, paranoia, and other conditions mediated by the CB2 receptor or other biological pathways.

### BRIEF DESCRIPTION OF THE DRAWINGS

FIG. 1A represents structures of illustrative prenylated cannabinoid compounds and analog scaffolds with variable alkyl side chain lengths and FIG. 1B represents the structure of prenylated cannabinoids having a 5-propylbenzene-1,3-diol core.

FIG. 2 shows GC×GC-TOF-MS chromatograms of cannabis Sample 1 (top) and synthesized SCBG reference standard (bottom).

FIG. 3 shows mass spectra of SCBG sample and reference standard plotting intensity as a function of mass-to-charge ratio to confirm the identity of sesqui-cannabigerol.

FIG. 4 shows GC×GC-TOF-MS chromatograms of cannabis Sample 26 (top) and synthesized SCBGV reference standard (bottom)

FIG. 5 shows mass spectra of SCBGV sample and reference standard plotting intensity as a function of mass-to-charge ratio to confirm the identity of sesqui-cannabigerovarin.

FIG. 6 illustrates a synthetic reaction scheme depicting acid-catalyzed coupling of substituted benzene-1,3-diol precursors with sesquiterpene alcohol precursors to form prenylated cannabinoid products with variable alkyl substituents.

FIG. 7 represents plots illustrating correlation coefficients between SCBG and other analytes across multiple sample categories.

FIGS. 8A and 8B are a set of plots illustrating distributions of sesqui-cannabigerol concentration across terpene-defined sample groups, including a box plot (FIG. 8A) comparing low and high terpinolene samples, and a scatter plot (FIG. 8B) showing SCBG concentrations in terpinolene-rich varieties.

FIGS. 9A and 9B are a set of plots illustrating Principal Component Analysis (PCA) of Analysis of Variance (ANOVA) features for classifying high- and low-terpinolene varieties, wherein FIG. 9A displays data labeled according to terpinolene levels and FIG. 9B displays data labeled according to dominant terpene categories.

FIGS. 10A and 10B are plots of % cell viability vs. concentration (μM) for various tumor cells treated with SCBG compositions.

### DETAILED DESCRIPTION

### Definitions

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified. As a non-limiting example, a reference to “X and/or Y” can refer, in one embodiment, to X only (optionally including elements other than Y); in another embodiment, to Y only (optionally including elements other than X); in yet another embodiment, to both X and Y (optionally including other elements).

Unless indicated otherwise, reference to “percent” is to be understood as “weight percent,” and reference to “ratio” is as a weight/weight ratio.

As used herein, the term “terpene compound” is understood to mean any organic compound that contains at least one isoprene subunit within its structure and their oxygenated derivatives.

As used herein, the terms “odor” and “aroma” are used interchangeably and represent the sensory attributes of certain substances perceptibly determined by the olfactory system.

As used herein, the term “flavoring agent” is understood to mean an additive that is meant to improve the taste or aroma impression of food or other substances, and can include both natural and synthetic ingredients.

As used herein, the term “gassy” is understood to be an organoleptic descriptor that represents a type of Cannabis aroma that emulates a sulfurous petroleum odor.

As used herein, the term “strain” is used to represent various chemovars or cultivars.

As used herein, the term “cultivar” represents a group of similar plants that by structural features and performance (i.e., morphological and physiological characteristics) can be identified from other varieties within the same species. Furthermore, the term “cultivar” variously refers to a variety, strain, or race of plant that has been produced by horticultural or agronomic techniques and is not normally found in wild populations. The terms cultivar, variety, strain, and race are often used interchangeably by plant breeders, agronomists, and farmers.

As used herein, the term “prenylated cannabinoids” represents cannabinoid compounds that contain a prenyl (3,3-dimethylallyl) or related isoprenoid group attached to their molecular structure.

As used herein, the term “sesqui-cannabigerol” or SCBG represents a prenylated analog of cannabigerol (CBG) with a farnesyl group (a 15-carbon isoprenoid chain) attached to the benzene ring versus the typical geranyl group found in CBG.

As used herein, the term “sesqui-cannabigerolic acid” or SCBGA represents the acidic form of SCBG, containing a carboxylic acid group.

As used herein, the term “sesqui-cannabigerovarin” or SCBGV represents a variant of SCBG with a shorter alkyl side chain (propyl instead of pentyl).

As used herein, the term CB1 and CB2 Receptors represent cannabinoid receptors in the endocannabinoid system, where CB1 is primarily in the central nervous system and CB2 is associated with the immune system.

The term “endogenous” as used herein refers to a component which is present and/or naturally expressed within a plant or a cell thereof. As used herein, the term “exogenous” refers to a component which may not be present and/or naturally expressed within the plant or cell thereof, e.g., a component which may be externally added.

As used herein, a “cancer” is a disease state characterized by the presence in a subject of cells demonstrating abnormal uncontrolled replication and in some aspects, the term may be used interchangeably with the term “tumor.” In some embodiments, the cancer is a solid tumor. In other embodiments, the cancer is not a solid tumor. In some embodiments, the cancer is from a carcinoma, a sarcoma, a myeloma, a leukemia, or a lymphoma. In some embodiments, the cancer is a primary cancer or a metastatic cancer. In some embodiments, the cancer is a relapsed cancer. In some embodiments, the cancer reaches a remission, but can relapse. In some embodiments, the cancer is unresectable.

The present technology offers a comprehensive solution to enhancing the properties of SCBG and related prenylated cannabinoids. By providing novel formulations at efficacious concentrations and methods for their synthesis or extraction, the present technology expands the chemical landscape of cannabinoids and opens new pathways for therapeutic interventions targeting the CB2 receptor.

### Prenylated Cannabinoid Compounds

Various aspects and/or embodiments of this disclosure are related to novel compositions and methods involving prenylated cannabinoids, specifically sesqui-cannabigerol (SCBG), its acidic analog SCBGA, sesqui-cannabigerovarin (SCBGV), and SCBGVA, and other prenylated cannabinoids. Particularly, novel prenylated cannabinoids of the present technology include, without limitation, Sesquicannabigerol (SCBG) and related prenylated variants, Sesquicannabigerovarin (SCBG-varin), their acidic derivatives, their methyl ester derivatives and deuterated forms thereof. SCBG is a cannabinoid scaffold with an additional prenyl group on its unsaturated tail, SCBG-varin is an analog with a modified alkyl chain, their acidic derivatives include compounds in their carboxylic acid forms, and their methyl ester derivatives include esterified forms that may enhance lipophilicity and bioavailability. Deuterated forms of the prenylated cannabinoids can also be used.

These compounds offer a promising solution due to their potent CB2 receptor affinity, potentially reduced off-target effects (e.g., limited CB1 activity), and additional interactions with receptors such as TRPV, which may modulate both cancer cell behavior and the surrounding microenvironment. Additionally, these compounds exhibit strong affinity for the cannabinoid CB2 receptor and have significant therapeutic potential, particularly aiming at reducing anxiety and paranoia without intoxicating effects. The present technology includes methods for synthesizing these compounds and formulating them into various products with enhanced bioavailability and efficacy.

In one aspect, provided herein are prenylated cannabinoid compounds having a cannabigerol core substituted with a farnesyl or geranylgeranyl isoprenoid side chain, or a pharmaceutically acceptable salts thereof, deuterated forms thereof, esters thereof, acidic derivatives thereof, metabolites thereof, or functionalized derivative thereof. Prenylated cannabinoids as mentioned herein are meant to include the prenylated cannabinoid compounds, acids thereof, deuterated forms thereof, pharmaceutical salts thereof, esters thereof, other derivatives or metabolites thereof. In some embodiments, the prenylated cannabinoid comprises a saturated prenylated cannabinoid, a hydrogenated or a fluorinated prenylated cannabinoid.

In one aspect, provided herein are prenylated cannabinoid having the following formula (I):

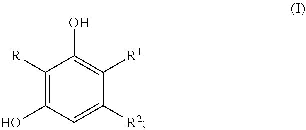

or a pharmaceutically acceptable salt, ester, or acidic derivative thereof; where R at each occurrence is a farnesyl group or a geranylgeranyl group; R¹ at each occurrence is hydrogen or —COOR³; R² at each occurrence is —C₃-C₂₀ alkyl; and R³ at each occurrence is selected from hydrogen, —C₁-C₂₀ alkyl, and —C₁-C₂₀ haloalkyl.

In some embodiments, the compound of Formula (I) is a compound R at each occurrence is a farnesyl group. In some embodiments, the compound of Formula (I) is a compound wherein R at each occurrence is a geranylgeranyl group. In some embodiments, the compound of Formula (I) is a compound R at each occurrence has C₁₅-C₂₀ isoprene units. In some embodiments, the compound of Formula (I) is a compound R at each occurrence has C₁₅ isoprene unit. In some embodiments, the compound of Formula (I) is a compound R at each occurrence has a C₂₀ isoprene unit.

In some embodiments, the compound of Formula (I) is a compound wherein R¹ is hydrogen. In some embodiments, the compound of Formula (I) is a compound wherein R¹ is-COOR³. In some embodiments, the compound of Formula (I) is a compound wherein R¹ is-COOH.

In some embodiments, the compound of Formula (I) is a compound wherein R² is-C₃-C₂₀ alkyl. In some embodiments, the compound of Formula (I) is a compound wherein R² is independently, methyl, ethyl, n-propyl, isopropyl, n-butyl, isobutyl, sec-butyl, tert-butyl, n-pentyl, isopentyl, neopentyl, tert-pentyl, n-hexyl, 1-methylpentyl, 4-methyl-2-pentyl, 3,3-dimethylbutyl, 2-ethylbutyl, n-heptyl, 1-methylhexyl, n-octyl, tert-octyl, 1-methylheptyl, 2-ethylhexyl, 2-propylpentyl, n-nonyl, 2,2-dimethylheptyl, 2,6-dimethyl-4-heptyl, 3,5,5-trimethylhexyl, n-decyl, n-undecyl, 1-methyldecyl, n-dodecyl, n-tridecyl, 1-hexylheptyl, n-tetradecyl, n-pentadecyl, n-hexadecyl, n-heptadecyl, n-octadecyl, and n-icosyl, n-nonadecyl, or n-docadecyl. In some embodiments, the compound of Formula (I) is a compound wherein R² is n-propyl. In some embodiments, the compound of Formula (I) is a compound wherein R² is n-butyl. In some embodiments, the compound of Formula (I) is a compound wherein R² is n-pentyl. In some embodiments, the compound of Formula (I) is a compound wherein R² is n-heptyl.

In some embodiments, the compound of Formula (I) is a compound wherein R³ is hydrogen. In some embodiments, the compound of Formula (I) is a compound wherein R³ isc-C₁-C₂₀ alkyl. In some embodiments, the compound of Formula (I) is a compound wherein R³ is-C₁-C₂₀ haloalkyl.

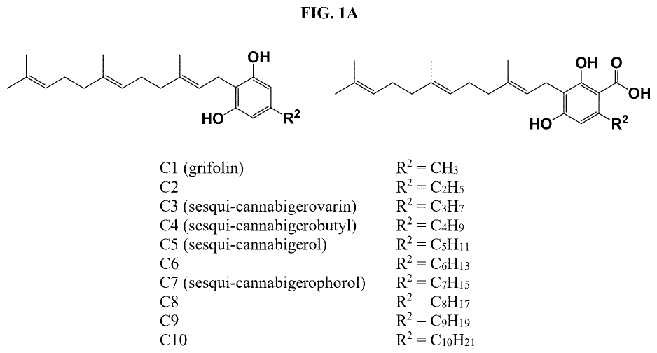

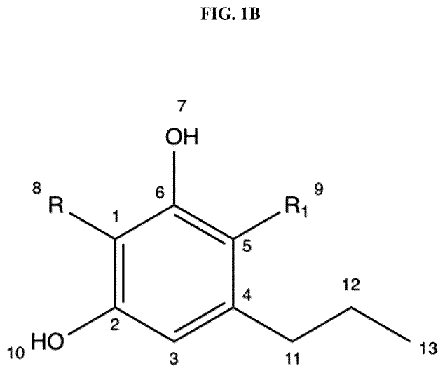

In one aspect, provided herein are prenylated cannabinoid compounds having a cannabigerol core substituted with a farnesyl or geranylgeranyl isoprenoid side chain. In some embodiments the prenylated cannabinoid comprises SCBG, SCBGB, SCBGP, SCBGG, SCBGVA, SCBGBA, SCBGA, SCBGPA, SCBGGA, deuterated forms thereof, pharmaceutical salts thereof, esters thereof, or metabolites thereof. Structures of illustrative prenylated cannabinoid compounds and analogs are shown in FIGS. 1A and 1B. Illustrative compounds of Formula (I) are summarized in Table 1, below.

TABLE 1

| | Abbreviated | | | |
|---|---|---|---|---|
| Compound Name | Name | R | R¹ | R² |
| sesqui- | SCBGV | farnesyl | H | n-propyl |
| cannabigerovarin | | | | |
| sesqui- | SCBGB | farnesyl | H | n-butyl |
| cannabigerobutyl | | | | |
| sesqui-cannabigerol | SCBG | farnesyl | H | n-pentyl |
| sesqui- | SCBGP | farnesyl | H | n-heptyl |
| cannabigerophorol | | | | |
| sesqui- | SCBGG | geranylgeranyl | H | n-pentyl |
| cannabigeranylgerol | | | | |
| sesqui- | SCBGVA | farnesyl | COOH | n-propyl |
| cannabigerovarinic | | | | |
| acid | | | | |
| sesqui- | SCBGBA | farnesyl | COOH | n-butyl |
| cannabigerobutyric | | | | |
| acid | | | | |
| sesqui- | SCBGA | farnesyl | COOH | n-pentyl |
| cannabigerolic | | | | |
| acid | | | | |
| sesqui- | SCBGPA | farnesyl | COOH | n-heptyl |
| cannabigerophorolic | | | | |
| acid | | | | |
| sesqui- | SCBGGA | geranylgeranyl | COOH | n-pentyl |
| cannabigeranyl- | | | | |
| gerolic acid | | | | |

In one aspect, provided is a compound which is sesqui-cannabigerol (SCBG) having the following formula:

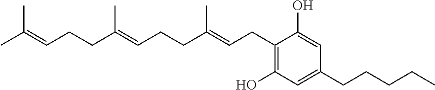 [SMILES: CCCCCc1cc(O)c(C/C=C(\C)CC/C=C(\C)CCC=C(C)C)c(O)c1]

or a pharmaceutically acceptable salt, ester, or acidic derivative thereof.

In one aspect, provided is a compound which is sesqui-cannabigeranylgerol (SCBGG) having the following formula:

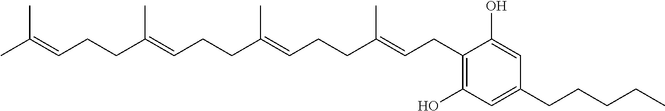 [SMILES: CCCCCc1cc(O)c(C/C=C(\C)CC/C=C(\C)CC/C=C(\C)CCC=C(C)C)c(O)c1]

or a pharmaceutically acceptable salt, ester, or acidic derivative thereof. In one aspect, provided is a compound which is sesqui-cannabigerovarin (SCBGV) having the following formula:

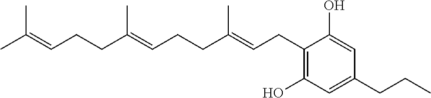 [SMILES: CCCc1cc(O)c(C/C=C(\C)CC/C=C(\C)CCC=C(C)C)c(O)c1]

or a pharmaceutically acceptable salt, ester, or acidic derivative thereof.

In one aspect, provided is a compound which is sesqui-cannabigerobutyl (SCBGB) having the following formula:

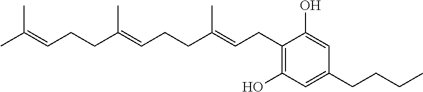 [SMILES: CCCCc1cc(O)c(C/C=C(\C)CC/C=C(\C)CCC=C(C)C)c(O)c1]

or a pharmaceutically acceptable salt, ester, or acidic derivative thereof.

In one aspect, provided is a compound which is sesqui-cannabigerophorol (SCBGP) having the following formula:

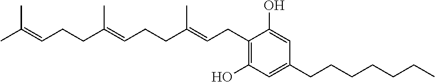 [SMILES: CCCCCCCc1cc(O)c(C/C=C(\C)CC/C=C(\C)CCC=C(C)C)c(O)c1]

or a pharmaceutically acceptable salt, ester, or acidic derivative thereof.

In one aspect, provided is a compound which is sesqui-cannabigerolic acid (SCBGA) having the following formula:

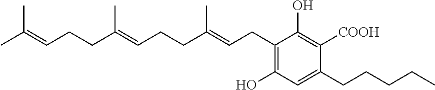 [SMILES: CCCCCc1cc(O)c(C/C=C(\C)CC/C=C(\C)CCC=C(C)C)c(O)c1C(=O)O]

or a pharmaceutically acceptable salt, ester, or acidic derivative thereof.

In one aspect, provided is a compound which is sesqui-cannabigeranylgerolic acid (SCBGGA) having the following formula:

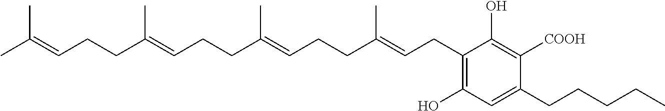 [SMILES: CCCCCc1cc(O)c(C/C=C(\C)CC/C=C(\C)CC/C=C(\C)CCC=C(C)C)c(O)c1C(=O)O]

or a pharmaceutically acceptable salt, ester, or acidic derivative thereof. In one aspect, provided is a compound which is sesqui-cannabigerovarinic acid (SCBGVA) having the following formula:

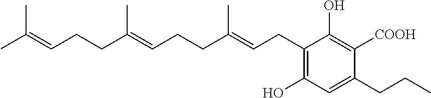 [SMILES: CCCc1cc(O)c(C/C=C(\C)CC/C=C(\C)CCC=C(C)C)c(O)c1C(=O)O]

or a pharmaceutically acceptable salt, ester, or acidic derivative thereof.

In one aspect, provided is a compound which is sesqui-cannabigerobutyric (SCBGBA) having the following formula:

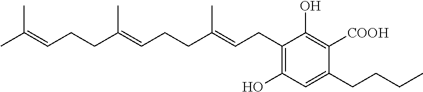 [SMILES: CCCCc1cc(O)c(C/C=C(\C)CC/C=C(\C)CCC=C(C)C)c(O)c1C(=O)O]

or a pharmaceutically acceptable salt, ester, or acidic derivative thereof.

In one aspect, provided is a compound which is sesqui-cannabigerophorolic acid (SCBGPA) having the following formula:

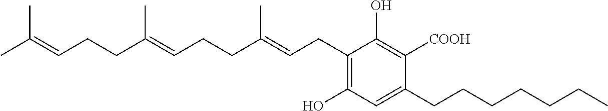 [SMILES: CCCCCCCc1cc(O)c(C/C=C(\C)CC/C=C(\C)CCC=C(C)C)c(O)c1C(=O)O]

or a pharmaceutically acceptable salt, ester, or acidic derivative thereof.

The present inventors made the unexpected discovery of SCBG, its acidic analog SCBGA, and SCBGV—the varin analog of SCBG—in high-THC or high-THCV cannabis, respectively. SCBG is found among all samples measured, albeit at very low, unusable concentrations. The confirmation of SCBGA suggests that the acidic form is the biosynthetic precursor to SCBG. The present inventors also detected SCBGV in a refined high-THCV extract, indicating that variations in the alkyl side chain length, as observed in typical cannabinoids, also apply to these prenylated analogs.

Additionally, it has been discovered that high-terpinolene-rich cannabis varieties—typically classified as “sativa” and often anecdotally associated with increased paranoia or anxiety—have lower levels of SCBG relative to other varieties with different dominant terpenes. Strong CB2 receptor affinity has been confirmed via molecular docking studies for SCBG type compounds with alkyl side chain lengths ranging from one to ten carbons, including their acidic forms. Given this affinity for the CB2 receptor, this suggests that SCBG may modulate the effects of different cannabis strains, potentially influencing user experience and therapeutic outcomes, indicating that the disclosed compounds may possess significant therapeutic benefits. It has been found that the compounds disclosed herein can modulate cancer-related pathways, particularly via one or more of: (a) high affinity binding to the CB2 Receptor: potent CB2 activity is linked with anticancer effects, including the inhibition of proliferation and induction of apoptosis; (b) selective targeting over CB1: reduced engagement with CB1 receptors minimizes central nervous system side effects; and (c) additional modulation of TRPV channels and other targets: TRPV receptor affinity potentially contributing to tumor microenvironment modulation; and (d) additional targets: with potential interactions with and/or modulation of PPARγ and Akt kinase, contribute to the overall anticancer activity.

In another aspect, provided herein are prenylated cannabinoids that are non-naturally occurring or synthetically derived. In another aspect, provided herein are methods of synthesizing are prenylated cannabinoids, wherein the method includes providing an olivetol or olivetol derivative; reacting the olivetol or derivative with farnesyl bromide or geranylgeranyl bromide in the presence of a base to form a prenylated cannabinoid; and isolating and purifying the prenylated cannabinoids. In some embodiments, the base is potassium carbonate and the reaction in (b) is carried out in acetone at room temperature. In some embodiments, the method further includes converting the prenylated cannabinoid to its acidic form by carboxylation.

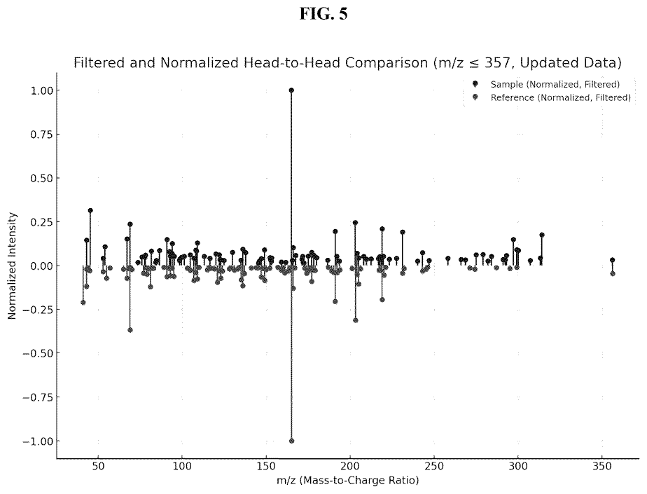

For example, the prenylated cannabinoids can be chemically synthesized as shown in the synthetic scheme in FIG. 6. General procedure is described in, for example, Jentsch N G, et al., J. Nat. Prod. 2020, 83 (9), 2587-2591. Typically, the phenolic precursor is added to a solution containing farnesol in DCE in a 1:1 ratio. Acidic Alumina is then added (4%), followed by reflux for 5 hours. The solution is then filtered, DCE evaporated, and resulting crude product purified using column chromatography. The crude products are analyzed using GC×GC-TOF-MS which confirmed their similar elution to SCBG in high-THC cannabis and SCBGV in high-THCV cannabis. Additional compounds can be similarly synthesized, including the C4 and C7 analogs, sesqui-cannabigerobutyl (SCBGB) and sesquicannabigerophorol (SCBGP), and the novel C5 analog with the geranylgeranyl unsaturated sidechain sesqui-cannabigeranylgerol (SCBGG), as well as their acid counterparts. In some embodiments, the SCBG is produced by a process comprising synthesizing SCBG from olivetol or an olivetol derivative and farnesyl bromide through a prenylation reaction. In some embodiments, the synthesis process may further include purification steps resulting in prenylated cannabinoids with a purity greater than about 90%, greater than about 95%, greater than about 98% or greater than about 99%, or any range or value including and/or in-between any two of these values.

In some embodiments, the prenylated cannabinoid exhibits higher or preferential binding affinity for CB1 and CB2 receptors. In some embodiments, the prenylated cannabinoid exhibits higher or preferential binding affinity for CB2 receptor relative to CB1 receptor. In some embodiments, the prenylated cannabinoid exhibits selective binding affinity for CB2 receptor. In some embodiments, the prenylated cannabinoid demonstrates selective binding to CB2 receptor over CB1 receptor, as evidenced by comparative docking data. In some embodiments, the prenylated cannabinoid exhibits high binding affinity to CB2 receptor, wherein the affinity is demonstrated by in silico docking studies showing a binding energy of −9 kcal/mol or higher.

### Compositions of Prenylated Cannabinoids

In one aspect, provided herein are compositions comprising one or more prenylated cannabinoids in an amount effective to modulate cannabinoid receptor CB2 activity; wherein the prenylated cannabinoid has the following formula (I), or a pharmaceutically acceptable salt, ester, or acidic derivative thereof. In some embodiments, the one or more prenylated cannabinoids is non-naturally occurring or synthetically derived. In some embodiments, the one or more prenylated cannabinoids are added exogenously to the composition and are not present solely as a naturally occurring component of cannabis or other natural product. In some embodiments, the composition is substantially free of other naturally occurring cannabinoids.

In one aspect, provided herein are compositions comprising one or more prenylated cannabinoids selected from sesqui-cannabigerol (SCBG), sesqui-cannabigerovarin (SCBGV), sesqui-cannabigerobutyl (SCBGB), sesqui-cannabigerophorol (SCBGP) sesqui-cannabigeranylgerol (SCBGG), acidic derivatives thereof, alkyl ester derivatives thereof, or a combination of any two or more thereof. In one embodiment, the prenylated cannabinoid includes sesqui-cannabigeranylgerol (SCBGG). In one embodiment, the prenylated cannabinoid includes sesqui-cannabigerol (SCBG).

Prenylated cannabinoids such as sesqui-cannabigerol (SCBG) and its analogs can be formulated into various matrices or mixtures to enhance their efficacy and bioavailability. Due to their potential anxiolytic characteristics, these compounds may find particular utility in combination with cannabis or psychedelic substances to modulate and minimize anxiety or stress that may be induced by other active ingredients. Additionally, for general anxiety or stress management, prenylated cannabinoids hold strong potential for use in nutraceuticals aimed at alleviating these issues. In particular, stress-induced insomnia and related ailments may benefit greatly from the incorporation of prenylated cannabinoids. Thus in one embodiment, the compositions described herein are formulated as a nutraceutical product aimed at reducing insomnia including but not limited to stress-induced insomnia. In one embodiment, the compositions described herein are formulations of prenylated cannabinoids for administration through oral, nasal, topical, inhaled, or transdermal routes.

The concentration of prenylated cannabinoids in these compositions can range from about 0.01% to about 99%, including from about 0.1% to about 95%, from about 0.1% to about 90%, or from about 0.1% to about 50%, by weight, and values and ranges within and including these values, allowing for flexibility in dosing and formulation design. In some embodiments, the composition may include about 0.01% to about 99% of the prenylated cannabinoid compound of the total weight of the composition, including without limitation, about 0.01% to about 95%, about 0.05% to about 90%, about 0.1% to about 75%, about 0.5% to about 50%, about 1% to about 30%, about 5% to about 25%, or about 10% to about 15% by weight of the total weight of the composition, or any range including and/or in-between any two of these values. In some embodiments, the composition may include at least about 0.001% of the one or more of the prenylated cannabinoid compound by weight of the total weight of the composition, including at least about 0.002%, at least about 0.005%, at least about 0.01%, at least about 0.02%, at least about 0.03%, at least about 0.05%, at least about 0.1%, at least about 0.5%, at least about 1% or at least about 2% by weight. In some embodiments, the composition may include up to about 30% of the one or more of the prenylated cannabinoid by weight of the total composition, including up to about 90%, up to about 80%, up to about 70%, up to about 60%, up to about 50%, up to about 40%, up to about 30%, up to about 20%, up to about 10%, up to about 5%, up to about 2%, up to about 1% or up to about 0.1% by weight of the total weight of the composition. In some embodiments, the composition includes at least 0.001% by weight of the one or more prenylated cannabinoids by weight of the total composition. In some embodiments, the composition includes about 0.001% to about 1% by weight of the one or more prenylated cannabinoids by weight of the total composition. In some embodiments, the prenylated cannabinoid content in the composition is between 0.001% and 0.1% by weight of the total weight of the composition.

In some embodiments, the composition includes one or more prenylated cannabinoids is present at a concentration higher than its natural abundance in cannabis plants. For example, although SCBG is found in natural cannabis samples, the concentrations even in those with the highest amounts are relatively low (<10 ppm). While this may be enough to subtly differentiate terpinolene-rich varieties from other types, it limits the utility of this and the other compounds to harness their full potential. As such, formulations outside of the context of cannabis containing enriched amounts of prenylated cannabinoids can help further enhance the properties of these compounds. The present technology provides a solution by providing compositions comprising prenylated cannabinoids at concentrations exceeding natural abundance levels and enabling preparation of formulations In some embodiments, the composition includes one or more prenylated cannabinoids which are non-naturally occurring or synthetically derived.

For administration to a mammal, the compositions can include from about 1.0 to about 1000 milligrams of the one or more of the prenylated cannabinoid compound, including, without limitation about 0.01, 0.1, 0.5, 1.0, 3.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the one or more of the prenylated cannabinoid compound. In some embodiments, the composition includes between 0.1 mg and 500 mg of the one or more prenylated cannabinoids per unit dose.

Compositions of prenylated cannabinoids can include one or more pharmaceutically or nutraceutically acceptable carrier, optionally wherein the carrier is selected from the group consisting of oils, solvents, emulsifiers, and polymers.

Compositions of prenylated cannabinoids can be produced with a multitude of other active or inactive ingredients, depending on the intended application. Suitable active ingredients are described herein and include, but are not limited to, other cannabinoids, such as for example tetrahydrocannabinol (THC): may enhance therapeutic effects in certain applications; CBD: known for its anxiolytic and anti-inflammatory properties; CBG: offers potential antibacterial and neuroprotective benefits; CBN: associated with sedative effects, useful in sleep aids; CBC: may contribute anti-inflammatory and analgesic effects; THCV: being researched for appetite suppression and antidiabetic effects; CBDV: potentially beneficial in epilepsy treatment; CBGV: similar properties to CBG but with a propyl side chain; acidic variants of cannabinoids, and the like and combinations of any two or more thereof. In some embodiments, the prenylated cannabinoid composition further includes at least one additional cannabinoid selected from the group consisting of CBD, CBG, CBN, THC, and THCV, acidic forms thereof, or a combination of any two or more thereof. In some embodiments, the prenylated cannabinoid composition and additional cannabinoid are present in a ratio effective to synergistically modulate CB2 receptor activity. In some embodiments, the prenylated cannabinoid composition further includes CBD. In some embodiments, the prenylated cannabinoid composition includes SCBG and CBD, wherein the SCBG and CBD are present in a ratio effective to synergistically modulate CB2 receptor activity.

In some embodiments, the prenylated cannabinoid composition further includes one or more of a terpene selected from the group consisting of myrcene, limonene, linalool, beta-caryophyllene, and/or other terpenes and terpenoids. In some embodiments, the prenylated cannabinoid composition further includes one or more of a flavorant or blend thereof including but not limited to flavorant alcohols, esters, ketones, aldehydes, carboxylic acids, or heterocycles. In some embodiments, the prenylated cannabinoid composition further includes a terpene selected from the group consisting of myrcene and limonene.

The amount of active or inactive additive or ingredient in the composition can be determined based on the end use or application of the composition. For example, the compositions may include the active and/or inactive ingredient in an amount of about 0.01 to about 99.9 percent by weight, of the total weight of the composition, including at least about 5% by weight of the total composition, at least about 10%, at least about 20%, at least about 30%, at least about 40%, at least about 50%, at least about 60%, at least about 70%, at least about 80% or at least about 90% by weight. In certain embodiments, the active and/or inactive ingredient is present at up to about 99.99% by weight of the total composition, up to about 99%, up to about 95%, up to about 92%, up to about 90%, up to about 85% or up to about 80% by weight of the total weight of the composition. In certain embodiments, the active and/or inactive ingredient constitutes about 5% to about 99. 99% of the total weight of the composition, including without limitation, about 10% to about 98%, about 20% to about 98%, about 30% to about 98%, about 40% to about 98%, about 50% to about 95%, about 60% to about 90%, or about 70% to about 85% of the total weight of the composition, or any range including and/or in-between any two of these values.

The present technology further provides formulations containing the aforementioned compounds in pharmaceutically acceptable carriers. The present technology provides a pharmaceutical composition comprising a prenylated cannabinoid and a pharmaceutically acceptable carrier. The present technology provides a nutraceutical composition comprising a prenylated cannabinoid and a nutraceutically acceptable carrier. These compositions may be the formulated for oral, topical, inhalable, sublingual, buccal, transdermal, intravenous, or localized administration. In some embodiments, the compositions may be administered orally, intravenously, or via localized delivery methods optimized for cancer treatment.

The prenylated cannabinoids may be synthetically prepared using the methods described herein, and then optionally combined with other ingredients or additives described herein. The synthetic methodology enables production of prenylated cannabinoids in quantities exceeding the trace amounts obtainable through extraction from cannabis plant material, addressing the limitation posed by the extremely low natural abundance of these compounds and enabling preparation of formulations containing concentrations of prenylated cannabinoids higher than those found in natural cannabis extracts. Further, the prenylated cannabinoid obtained through the synthetic methods of the present technology may provide high purity compounds, e.g., greater than ninety-five percent, following isolation and purification, thereby providing material suitable for formulation into compositions comprising other ingredients such as pharmaceutically and nutraceutically acceptable carriers, other additive and ingredients, and other cannabinoid, hop or hemp materials. In some embodiments, the composition may include additives or ingredients such as hop oil, cannabis oil, hemp oil, hop extract, carrier oil, flavorant blends, terpene and terpenoids, organosulfur compounds, emulsifiers, hop-derived flavorant compounds, non-hop derived flavorant compounds, or a mixture of any two or more thereof. In some embodiments, the composition may include flavoring agents, sweeteners, and preservatives suitable for consumption.

Other ingredients for inclusion in the composition are described herein and may include, but are not limited to, terpenes and terpenoids such as myrcene: may enhance cannabinoid absorption; possesses sedative effects, limonene: known for uplifting mood and stress relief, linalool: exhibits calming and anxiolytic properties, pinene: may improve alertness and memory retention, beta-caryophyllene: acts as a CB2 receptor agonist; anti-inflammatory, humulene: potential anti-inflammatory and appetite suppressant effects, terpinolene: contributes to uplifting effects; antioxidant properties, and the like and combinations of any two or more thereof. Flavorants including commonly used flavorants such as ethyl hexanoate, ethyl butyrate, vanillin, etc.; botanical extracts and herbal supplements such as valerian root extract: traditionally used for sleep disorders, passionflower extract: may reduce anxiety and improve sleep quality, chamomile extract: known for its calming effects, kava extract: utilized for its anxiolytic properties, ashwagandha extract: an adaptogen that may help reduce stress; vitamins and minerals such as vitamin B complex: supports nervous system health, magnesium: may alleviate anxiety and improve sleep, zinc: contributes to immune function and neurotransmitter regulation, melatonin: regulates sleep-wake cycles and useful in sleep aids; amino acids and neurotransmitter precursors such as 1-theanine: promotes relaxation without drowsiness, gamma-aminobutyric acid (GABA): primary inhibitory neurotransmitter; reduces neuronal excitability, tryptophan: precursor to serotonin; may improve mood and sleep, psychedelic compounds (where legally permissible and under appropriate regulations), psilocybin: investigated for its potential in treating depression and anxiety, lysergic acid diethylamide (LSD): researched for microdosing benefits in mood regulation, n,n-dimethyltryptamine (dmt): studied for potential therapeutic effects, mescaline: explored for its psychoactive properties in mental health treatments; other therapeutic agents such as nonsteroidal anti-inflammatory drugs (NSAIDS): for pain relief, antioxidants: such as vitamins c and e to protect against oxidative stress, adaptogens: like Rhodiola rosea to enhance resistance to stress; inactive ingredients (excipients and carriers) such as oils and lipid carriers, medium-chain triglyceride (MCT) oil: enhances absorption of lipophilic compounds, hemp seed oil: provides essential fatty acids; compatible with cannabinoids, olive oil: common carrier oil with health benefits, coconut oil: used in edibles and topicals for its moisturizing properties, sunflower oil: a neutral carrier oil suitable for various formulations; solvents and co-solvents such as ethanol: used in tinctures and extraction processes, propylene glycol: common in vape liquids; solvent for oral solutions, glycerin (glycerol): humectant and sweetener in oral formulations; emulsifiers and surfactants such as lecithin: enhances bioavailability; used in liposomal formulations, polysorbate 80: stabilizes emulsions in aqueous solutions, sorbitan monooleate (span 80): used in emulsification processes, sodium lauryl sulfate: surfactant in topical formulations; polymers and matrix formers such as hydroxypropyl methylcellulose (HPMC): controlled-release agent in tablets, polyvinylpyrrolidone (PVP): binder and solubility enhancer, carbomers: thickening agents in gels and creams, gelatin: used in capsule shells and gummy formulations; sweeteners and flavoring agents such as natural sweeteners: stevia, monk fruit extract, honey, artificial sweeteners: sucralose, aspartame, flavorings: mint, citrus, berry, chocolate, vanilla; preservatives and stabilizers such as potassium sorbate: prevents microbial growth, sodium benzoate: preservative in liquid formulations, antioxidants such as butylated hydroxytoluene (BHT), butylated hydroxyanisole (BHA); fillers and binders such as microcrystalline cellulose: provides tablet hardness and structure, dicalcium phosphate: filler in tablets and capsules, starch: binder and disintegrant, lactose: common filler in solid dosage forms; disintegrants and flow aids such as croscarmellose sodium: facilitates tablet disintegration, silicon dioxide: improves powder flow properties, magnesium stearate: lubricant in tablet manufacturing; coating agents such as enteric coatings: cellulose acetate phthalate for delayed-release tablets, sugar coatings: improves taste and appearance; gelling agents and thickeners such as xanthan gum: stabilizes emulsions and used in oral and topical products, guar gum: thickener in suspensions and gels, agar: gelling agent in topical formulations, pectin: used in chewable and gummy formulations; humectants such as sorbitol: retains moisture; used in oral and topical products, glycerin: moisturizer in topicals and sweetener in orals; colorants such as natural colorants including beet extract, turmeric, chlorophyll, artificial dyes: FD&C approved colorants for pharmaceuticals.

The amount of other ingredients in the composition can be determined based on the end use or application of the composition. For example, the compositions may include one or more of the other ingredient in an amount of about 0.01 to about 99.9 percent by weight, of the total weight of the composition, including at least about 5% by weight of the total composition, at least about 10%, at least about 20%, at least about 30%, at least about 40%, at least about 50%, at least about 60%, at least about 70%, at least about 80% or at least about 90% by weight. In certain embodiments, the one or more of the other ingredient is present at up to about 99.99% by weight of the total composition, up to about 99%, up to about 95%, up to about 92%, up to about 90%, up to about 85% or up to about 80% by weight of the total weight of the composition. In certain embodiments, the one or more of the other ingredient constitutes about 5% to about 99. 99% of the total weight of the composition, including without limitation, about 10% to about 98%, about 20% to about 98%, about 30% to about 98%, about 40% to about 98%, about 50% to about 95%, about 60% to about 90%, or about 70% to about 85% of the total weight of the composition, or any range including and/or in-between any two of these values.

The compositions described herein may be formulated in any suitable form for administration or consumption. The pharmaceutical compositions containing the active ingredient (i.e., the prenylated cannabinoid) may be in a form suitable for oral use, for example, tablets, capsules, troches, lozenges, gummies, aqueous or oily suspensions, dispersible powders or granules, emulsions, hard or soft capsules, or syrups, solutions, microbeads, or elixirs. Pharmaceutical compositions intended for oral use may be prepared according to any method known to the art for the manufacture of pharmaceutical compositions, and such compositions may contain one or more agents such as, for example, sweetening agents, flavoring agents, coloring agents, and preserving agents in order to provide pharmaceutically elegant and palatable preparations. Tablets, capsules, and the like contain the active ingredient in admixture with non-toxic pharmaceutically acceptable excipients which are suitable for the manufacture of tablets. These excipients may be, for example, diluents, such as calcium carbonate, sodium carbonate, lactose, calcium phosphate, or sodium phosphate; granulating and disintegrating agents, for example, corn starch, or alginic acid; binding agents, for example starch, gelatin or acacia, and lubricating agents, for example magnesium stearate, stearic acid, or talc.

The suitable formulation of an oral dosage unit may also include, for example, a binder, such as, gum tragacanth, acacia, corn starch, gelatin; sweetening agents such as lactose or sucrose; disintegrating agents such as corn starch, alginic acid and the like; a lubricant such as magnesium stearate; or flavoring such a peppermint, oil of wintergreen or the like. Various other additives may be present as coating or to otherwise modify the physical form of the oral dosage unit. Any material utilized should be pharmaceutically-acceptable and substantially non-toxic. Details of the types of excipients useful may be found in the twenty first edition of “Remington: The Science and Practice of Pharmacy,” Mack Printing Company, Easton, PA. Some of the representative formulations and additives are described herein.

Tablets and capsules for oral administration can be uncoated or coated to control release in the gastrointestinal tract. Sustained action may use time-delay materials like glyceryl monostearate. Coatings and encapsulation methods include known polymeric substances and techniques such as microcapsules or colloidal systems (e.g., liposomes, microspheres) for controlled delivery. Oral formulations may be hard gelatin capsules with active ingredients mixed with inert fillers (e.g., calcium carbonate, kaolin) or soft gelatin capsules with water or oil bases (e.g., peanut oil, liquid paraffin). Aqueous suspensions mix active materials with excipients like suspending agents (celluloses, gums), wetting agents (lecithin, polyoxyethylene compounds), and may include preservatives. Oily suspensions use vegetable or mineral oils with possible thickening, sweetening, and flavoring agents for palatability. Dispersible powders and granules for aqueous suspensions contain active ingredients with dispersing, wetting, and suspending agents, and preservatives as described above.

The pharmaceutical compositions of the present technology may also be in the form of oil-in-water emulsions. The oily phase may be a vegetable oil, for example olive oil or arachis oil, or a mineral oil, for example, liquid paraffin, or mixtures of these. Suitable emulsifying agents may be naturally occurring gums, for example, gum acacia or gum tragacanth; naturally occurring phosphatides, for example, soybean, lecithin, and esters or partial esters derived from fatty acids; hexitol anhydrides, for example, sorbitan monooleate; and condensation products of partial esters with ethylene oxide, for example, polyoxyethylene sorbitan monooleate.

The pharmaceutical compositions may include a therapeutically effective amount of a prenylated cannabinoid contemplated by the present technology and one or more pharmaceutically and physiologically acceptable formulation agents. In some embodiments, the composition comprising a prenylated cannabinoid and a carrier solvent is provided for use in the treatment of cancer. In some embodiments, the carrier solvent is Dimethyl sulfoxide (DMSO).

In some embodiments, the composition may be formulated as a beverage, alcoholic beverage, herbal supplement, nutraceutical supplement, protein supplement, pre-workout supplement, ready to mix beverage powder, brewed beverage, fermented beverage, edible product, candy, baked good, food product, tincture, capsule, tablet, cream, lotion, or vape liquid. In some embodiments, the composition is formulated as a beverage containing between 1 mg and 100 mg of prenylated cannabinoids per serving. In some embodiments, the composition is a beverage composition comprising an effective amount of prenylated cannabinoids to modulate CB2 receptor activity, wherein the prenylated cannabinoids are present at a concentration higher than its natural abundance in cannabis plants.

Generally, the prenylated cannabinoids can be incorporated into various dosage forms, including but not limited to:
Oral Formulations:
   Capsules: Hard gelatin or vegetarian capsules containing oils or powders.
   Tablets: Immediate-release, sustained-release, or chewable tablets.
   Powders and Granules: For mixing with food or beverages.
   Oral Solutions and Suspensions: Liquid preparations for ease of administration.
   Gummies and Chewable: Palatable forms for consumer preference.
Topical Formulations:
   Creams and Lotions: For localized delivery through the skin.
   Gels: Fast-absorbing formulations for transdermal applications.
   Ointments: For prolonged contact and absorption.
   Transdermal Patches: Controlled release over extended periods.
Inhalable Formulations:
   Vape Oils and E-Liquids: For use in vaporizing devices.
   Dry Powder Inhalers: Micronized powders for pulmonary delivery.
   Sublingual and Buccal Formulations:
Tinctures: Alcohol or oil-based drops for rapid absorption.
   Sprays: Metered-dose applications for sublingual delivery.
   Oral Films: Dissolvable strips placed under the tongue.
Suppositories:
   Rectal and Vaginal Suppositories: For systemic or localized effects.
Beverages and Edibles:
   Infused Drinks: Teas, coffees, juices fortified with prenylated cannabinoids.
   Baked Goods: Cookies, brownies, and breads.
   Confections: Chocolates, hard candies, lollipops.
   Snack Bars: Nutrient-dense bars for convenience.

### Methods of Preparation of Formulations

Formulations can be prepared using standard pharmaceutical and nutraceutical processes. Mixing and blending techniques are employed to ensure uniform distribution of ingredients throughout the product. Granulation methods, either wet or dry, are used to form granules suitable for tableting, enhancing compressibility and flow properties. Encapsulation involves filling capsules with liquids, oils, or powders, providing a convenient dosage form. Compression techniques are utilized to form tablets under high pressure, ensuring consistent tablet weight and integrity. Emulsification creates stable oil-in-water or water-in-oil emulsions, which are essential for products like creams and lotions. Homogenization reduces particle size to achieve uniformity and stability in suspensions and emulsions. Spray drying is employed to produce dry powders from liquid feeds, useful for creating inhalable formulations or instant-mix products. Freeze drying, or lyophilization, is used to preserve sensitive compounds by removing moisture under low temperature and pressure, resulting in a stable, dry product that reconstitutes easily.

To enhance the bioavailability of prenylated cannabinoids, formulations may incorporate various strategies. Lipid-based delivery systems such as self-emulsifying drug delivery systems (SEDDS) enhance solubility and absorption of lipophilic compounds by forming fine oil-in-water emulsions upon contact with gastrointestinal fluids. Liposomes can encapsulate active ingredients within phospholipid bilayers for targeted delivery and sustained release. Nano-emulsions increase the surface area for absorption due to their small droplet size, improving the dissolution rate and bioavailability of the active compounds.

Permeation enhancers are also used to improve bioavailability. Piperine, for example, increases bioavailability by inhibiting metabolizing enzymes such as cytochrome P450 enzymes, thereby prolonging the active compound's presence in the system. Capsaicin may enhance transdermal absorption by increasing skin permeability, making it useful in topical formulations. In some embodiments, provided is a transdermal formulation comprising one or more prenylated cannabinoids and a permeation enhancer selected from the group consisting of but not limited to alpha bisabolol, piperine, limonene, and/or capsaicin.

Cyclodextrin complexes are employed to enhance solubility and stability. Beta-cyclodextrin forms inclusion complexes with hydrophobic molecules, effectively increasing their aqueous solubility. Hydroxypropyl beta-cyclodextrin, a modified form, further improves solubility and stability, making it suitable for injectable and oral liquid formulations.

Solid dispersions involve dispersing the active ingredient in a solid matrix, such as polymers or sugars, to enhance dissolution rates. This method increases the surface area of the active compound and can improve its wettability and solubility, leading to better bioavailability.

Formulations should be designed to protect prenylated cannabinoids from degradation due to environmental factors. To mitigate degradation from light exposure, opaque or amber-colored packaging is used to shield the product from harmful UV rays. Oxidation is addressed by including antioxidants such as butylated hydroxytoluene (BHT) or ascorbic acid in the formulation, and by using oxygen-impermeable packaging materials to prevent air ingress. Heat and humidity can accelerate degradation processes, so storage instructions are provided to maintain product integrity, typically recommending storage in cool, dry places away from direct sunlight. These measures ensure the stability of the formulation over its intended shelf life, preserving efficacy and safety for the end-user.

In one aspect, provided herein are methods of modulating CB2 receptor activity in a subject in need thereof, comprising administering to the subject, a therapeutically effective amount of the composition containing one or more prenylated cannabinoids of formula (I) in an amount effective to modulate cannabinoid receptor CB2 activity. In one aspect, provided herein is use of one or more prenylated cannabinoids of formula (I) in the manufacture of a medicament for treating conditions mediated by CB2 receptor activity. In some embodiments, the modulation of CB2 receptor activity results in a reduction of anxiety, paranoia, inflammation, pain, or a combination of any two or more thereof. In some embodiments, the subject is experiencing anxiety or paranoia associated with or induced by administration of tetrahydrocannabinol (THC) or psychedelic compounds. In some embodiments, the condition is selected from the group consisting of anxiety disorders, neuroinflammation, autoimmune diseases, and chronic pain. In some embodiments, the psychedelic compound is selected from the group consisting of psilocybin, LSD, DMT, and mescaline. In some embodiments, the prenylated cannabinoid is present in an amount effective to modulate anxiety or stress induced by the psychedelic compound. In some embodiments, the composition is formulated for oral administration in a controlled-release dosage form. In some embodiments, the prenylated cannabinoids are not encapsulated.

In one aspect, provided herein are methods of alleviating anxiety in a subject, comprising administering to the subject an effective amount of composition containing one or more prenylated cannabinoids of formula (I) sufficient to reduce or alleviate the symptoms of anxiety. In some embodiments, the prenylated cannabinoid is administered in combination with a pharmaceutically acceptable carrier and at least one additional anxiolytic agent.

In one aspect, provided herein are methods of treating cancer in a subject in need thereof, comprising administering an effective amount of the composition containing one or more prenylated cannabinoids of formula (I). In some embodiments, the cancer is breast cancer. In some embodiments, the cancer is triple-negative breast cancer. A method of treating cancer, and particularly TNBC, is provided by administering an effective amount of the prenylated cannabinoid composition to a subject in need thereof. Dosage forms and treatment regimens may be adjusted based on the pharmacokinetic properties elucidated through preclinical studies. In some embodiments, the methods include treating cancer by interaction of a prenylated cannabinoid and an exterior cell wall of a cancer cell. In some embodiments, the methods include treating cancer by interaction of a prenylated cannabinoid mediated through CB2 interactions.

The compounds, compositions and methods disclosed herein can be used to treat one or more of the following: acquired hypothyroidism, acute gastritis, agoraphobia, aids related illness, alcohol abuse, alcoholism, alopecia areata, Alzheimer's disease, amphetamine dependency, amyloidosis, amyotrophic lateral sclerosis (ALS), angina pectoris, ankylosis, ankylosing spondylitis, anorexia, anorexia nervosa, anxiety disorders, any chronic medical symptom that limits major life activities, any chronic medical symptom that limits major life activities, arteriosclerotic heart disease, arthritis, arthritis (rheumatoid), arthropathy, gout, asthma, attention deficit hyperactivity disorder (ADD/ADHD), autism, autoimmune disease, back pain, back sprain, Bell's palsy, bipolar disorder, brain tumor, malignant, bruxism, bulimia, cachexia, cancer, carpal tunnel syndrome, cerebral palsy, cervical disk disease, cervicobrachial syndrome, chemotherapy chronic fatigue syndrome, chronic pain, chronic renal failure, cocaine dependence, colitis, conjunctivitis, constipation, Crohn's disease, cystic fibrosis, damage to spinal cord nervous tissue, Darier's disease, degenerative arthritis, degenerative arthropathy, delirium tremens, dermatomyositis, diabetes, diabetic neuropathy, diabetic peripheral vascular disease, diarrhea, diverticulitis, dysthymic disorder, eczema, emphysema, emphysema, endometriosis, epidermolysis bullosa, epididymitis, epilepsy, Felty's syndrome, fibromyalgia, Friedreich's ataxia, gastritis, genital herpes, glaucoma, glioblastoma multiforme, Graves disease, cluster headaches, migraine headaches, tension headaches, hemophilia a, Henoch-Schonlein purpura, hepatitis C, hereditary spinal ataxia, HIV/AIDS, hospice patients, Huntington's disease, hypertension, hypertension, hyperventilation, hypoglycemia, impotence, inflammatory autoimmune-mediated arthritis, inflammatory bowel disease (IBD), insomnia, intermittent explosive disorder (IED), intractable pain, intractable vomiting, lipomatosis, Lou Gehrig's disease, lyme disease, lymphoma, major depression, malignant melanoma, mania, melorheostosis, Meniere's disease, motion sickness, mucopolysaccharidosis (MPS), multiple sclerosis (MS), muscle spasms, muscular dystrophy, myeloid leukemia, nail-patella syndrome, nightmares, obesity, obsessive compulsive disorder, opiate dependence, osteoarthritis, panic disorder, Parkinson's disease, peripheral neuropathy, peritoneal pain, persistent insomnia, porphyria, post-polio syndrome (PPS), post-traumatic arthritis, post-traumatic stress disorder (PTSD), premenstrual syndrome (PMS), prostatitis, psoriasis, pulmonary fibrosis, quadriplegia, radiation therapy, Raynaud's disease, Reiter's syndrome, restless legs syndrome (RLS), rheumatoid arthritis, rosacea, schizoaffective disorder, schizophrenia, scoliosis, sedative dependence, seizures, senile dementia, severe nausea, shingles (herpes zoster), sinusitis, skeletal muscular spasticity, sleep apnea, sleep disorders, spasticity, spinal stenosis, Sturge-Weber syndrome (SWS), stuttering, tardive dyskinesia (TD), temporomandibular joint disorder (TMJ), tenosynovitis, terminal illness, thyroiditis, tic douloureux, Tietze's syndrome, tinnitus, tobacco dependence, Tourette's syndrome, trichotillomania, viral hepatitis, wasting syndrome, whiplash, Wittmaack-Ekbom's syndrome, writers' cramp, nausea, vomiting, unintentional weight loss, insomnia, and lack of appetite, spasticity, painful conditions, especially neurogenic pain, movement disorders, asthma, glaucoma, adrenal disease, migraines, fibromyalgia, and related conditions, spinal cord injuries. The formulations disclosed herein can exhibit antispasmodic and muscle-relaxant properties as well as stimulates appetite, and may be useful in treating alcohol abuse, amyotrophic lateral sclerosis, collagen-induced arthritis, asthma, atherosclerosis, bipolar disorder, colorectal cancer, HIV-associated sensory neuropathy, depression, dystonia, epilepsy, digestive diseases, gliomas, hepatitis C, Huntington's disease, leukemia, skin tumors, methicillin-resistant Staphylococcus aureus (MRSA), Parkinson's disease, pruritus, posttraumatic stress disorder (PTSD), psoriasis, sickle-cell disease, sleep apnea, and anorexia nervosa.

The compounds, compositions and methods of the present technology can be used for the treatment of various types of cancer, and can be tested using various cancer cell lines know in the art. Illustrative cell lines are noted below:

Prostate Cancer: Cannabinoids can modulate proliferation and apoptosis in prostate cancer cells. Prostate cancer models often reveal cannabinoid-mediated regulation of androgen receptor signaling and cell cycle control. Examples: PC-3, DU145, LNCaP.

Colorectal Cancer: Several studies indicate that cannabinoids can inhibit colon cancer cell proliferation, induce cell cycle arrest, and promote apoptosis, possibly via modulating inflammatory pathways and angiogenesis. Examples: HCT116, HT29, SW480.

Lung Cancer: Lung cancer cell lines have been used to study the effects of cannabinoids on tumor cell migration, invasion, and apoptosis. These models help elucidate the modulation of key signaling pathways (e.g., MAPK/ERK, PI3K/Akt) by cannabinoids. Examples: A549, H1299.

Pancreatic Cancer: Cannabinoid compounds have shown potential in reducing tumor growth and metastasis in pancreatic cancer models. The aggressive nature of pancreatic tumors makes them a compelling target for new therapeutic agents. Examples: PANC-1, MiaPaCa-2.

Melanoma: Melanoma cells have been observed to undergo apoptosis and exhibit reduced metastatic potential upon cannabinoid treatment, which may be mediated through various receptor-dependent and independent mechanisms. Examples: A375, SK-MEL-28.

Other Hematological Malignancies (Leukemia/Lymphoma): Cannabinoids have demonstrated cytotoxic effects in some leukemia and lymphoma cell lines, making them worth exploring as part of a broader anticancer strategy. Examples: Jurkat (T-cell leukemia), HL-60 (promyelocytic leukemia).

Breast Cancer: Cannabinoids compounds are particularly interesting against cancers such as triple-negative breast cancer. Examples: MCF7, MDA-MB-231/ATCC, MDA-MB-468, HS 578T.

A non-exhaustive list of cancers includes bladder cancer, bone cancer, brain cancer, breast cancer, cervical cancer, colon cancer, esophageal cancer, gastric cancer, head and neck cancers, hodgkin's lymphoma, leukemia, liver cancer, lung cancer, melanoma, mesothelioma, multiple myeloma, myelodysplastic syndrome, non-hodgkin's lymphoma, ovarian cancer, pancreatic cancer, prostate cancer, rectal cancer, renal cancer, sarcoma, skin cancer, testicular cancer, thyroid cancer, etc.

In silico docking studies have been performed to evaluate the binding affinities and selectivity of the compounds. These studies indicate that the prenylated cannabinoids demonstrate high binding affinity to the CB2 receptor and favorable selectivity profiles over CB1. Additional binding to TRPV1 and other cancer-relevant targets supports their potential multi-modal anticancer activity.

The compounds, compositions and methods of the present technology have several advantages including, but not limited to improved safety profile due to the established tolerability of cannabinoids, potent anticancer activity through selective receptor modulation, multi-targeted approach addressing both tumor cell proliferation and the tumor microenvironment, and potential for combination therapy with other anticancer agents.

### EXAMPLES

Various embodiments will be further clarified by the following examples, which are in no way intended to limit this disclosure thereto.

### Example 1: Synthesis of SCBG

Sesquicannabigerol, 5-pentyl-2-((2E,6E)-3,7,11-trimethyldodeca-2,6,10-trienyl)benzene-1,3-diol. To a solution of farnesol (252 μL, 1.0 mmol) and olivetol (810 mg, 3.0 mmol) in dichloroethane (DCE) (10 mL) is added acidic alumina (2.0 g). The resulting heterogeneous mixture is stirred at reflux temperature for 7 h, then cooled to ambient temperature and filtered through 0.22 μm syringe filters. The filtrate is concentrated via rotary evaporation to yield a yellow oil. This oil is purified via flash column chromatography using silica gel eluted with hexanes and ethyl acetate. Product rich fractions are combined, and solvent is evaporated via rotary evaporation to yield sesquicannabigerinol.

### Example 2: Synthesis of SCBGA

Sesquicannabigerinolic acid, 5-pentyl-2-((2E,6E)-3,7,11-trimethyldodeca-2,6,10-trienyl)benzene-1,3-diol. To a solution of farnesol (252 μL, 1.0 mmol) and olivetolic acid (810 mg, 3.0 mmol) in dichloroethane (DCE) (10 mL) is added acidic alumina (2.0 g). The heterogeneous mixture is stirred at reflux temperature for 7 h, then cooled to ambient temperature and filtered through 0.22 μm syringe filters. The filtrate is concentrated via rotary evaporation to yield a yellow oil. This oil is purified via flash column chromatography using silica gel eluted with hexanes and EtOAc. Product rich fractions are combined, and solvent is evaporated via rotary evaporation to yield sesquicannabigerinolic acid.

### Example 3: Synthesis of SCBGV

Sesquicannabigerovarin, 5-pentyl-2-((2E,6E)-3,7,11-trimethyldodeca-2,6,10-trienyl)benzene-1,3-diol. To a solution of farnesol (252 μL, 1.0 mmol) and olivetolic acid (810 mg, 3.0 mmol) in dichloroethane (DCE) (10 mL) is added acidic alumina (2.0 g). The heterogeneous mixture is stirred at reflux temperature for 7 h, then cooled to ambient temperature and filtered through 0.22 μm syringe filters. The filtrate is concentrated via rotary evaporation to yield a yellow oil. This oil is purified via flash column chromatography using silica gel eluted with hexanes and EtOAc. Product rich fractions are combined, and solvent is evaporated via rotary evaporation to yield sesquicannabigerovarin.

### Example 4: Synthesis of SCBGG

Sesqui-cannabigeranylgerol, 5-pentyl-2-((2E,6E,10E)-3,7,11,15-tetramethylhexadeca-2,6,10,14-tetraen-1-yl)benzene-1,3-diol. To a solution of geranylgeraniol (3.25 mL, 9.9 mmol) and olivetol (5.41 g, 30.0 mmol) in dichloroethane (DCE) (100 mL) is added acidic alumina (20.0 g). The heterogeneous mixture is stirred at reflux temperature for 7 h, then cooled to ambient temperature and filtered through a Buchner funnel and filter pad under vacuum. The filtrate is concentrated via rotary evaporation to yield amber crude oil. This oil is purified via column chromatography using silica gel eluted with hexanes and EtOAc. Product rich fractions are combined, and solvent is evaporated via rotary evaporation and dried under vacuum to yield sesqui-cannabigeranylgerol.

### Example 5: Synthesis of SCBGP

Sesqui-cannabigerophorol, 5-heptyl-2-((2E,6E)-3,7,11-trimethyldodeca-2,6,10-trien-1-yl)benzene-1,3-diol. To a solution of farnesol (2.51 mL, 9.98 mmol) and olivetol (6.256 g, 30.0 mmol) in dichloroethane (DCE) (100 mL) is added acidic alumina (20.0 g). The heterogeneous mixture is stirred at reflux temperature for 7 h, then cooled to ambient temperature and filtered through a Buchner funnel and filter pad under vacuum. The filtrate is concentrated via rotary evaporation to yield a clear yellow crude oil. This oil is purified via column chromatography using silica gel eluted with hexanes and EtOAc. Product rich fractions are combined, and solvent is evaporated via rotary evaporation and dried under vacuum to yield sesqui-cannabigerophorol.

### Example 6: Analysis and Characterization of Prenylated Cannabinoids in Cannabis Samples

GC×GC-TOF-MS/FID. GC×GC analysis is performed using the INSIGHT reverse fill flush flow modulator (SepSolve Analytical). This is coupled with an Agilent 7890B GC equipped with a BPX5 (20 m×0.18 mm ID×0.18 μm film thickness) first dimension column and Mega Wax HT (4.8 m×0.32 mm ID×0.15 μm film thickness) second dimension column and BenchTOF2 Select mass spectrometer (Markes International). TOF-MS is used to identify the compounds. Quantification of all terpenes is done with FID. Quantification of prenylated cannabinoids is done using TOF-MS, with ions m/z=193 and 384. Sample introduction is done using direct injection with an Agilent 7693 Injector Tower (G4513A). The syringe is washed three times with isopropyl alcohol and hexanes before and after injection. The injection volume used is 3 μL. The inlet split flow and temperature are 20:1 and 280° C., respectively. The TOF-MS ion source is held at 280° C. and a transfer line temperature of 260° C. Mass spectral data are acquired at 50 Hz with a scan range of 40-400 m/z with a solvent delay of 6 min.

HPLC. Each flower sample is ground and homogenized using a grinder. 0.50 g (+-0.02 g) of ground sample is weighed into a 50 mL centrifuge tube. Extracts are added directly to the centrifuge tube. The tube is filled to the 50 mL mark with HPLC grade methanol, vortexed, and placed in an ultrasonication bath for 15 minutes. The sample is then vortexed and filtered through a 0.22 μm syringe filter. A 100 μL aliquot is taken and diluted with 900 μL HPLC grade methanol in an autosampler vial, and vortexed to mix. The prepared samples are analyzed using high-performance-liquid chromatography (HPLC) with a diode array detector (Agilent Technologies).

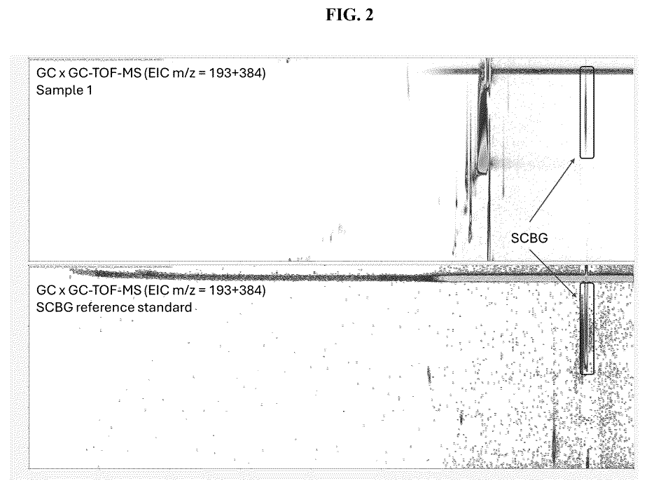

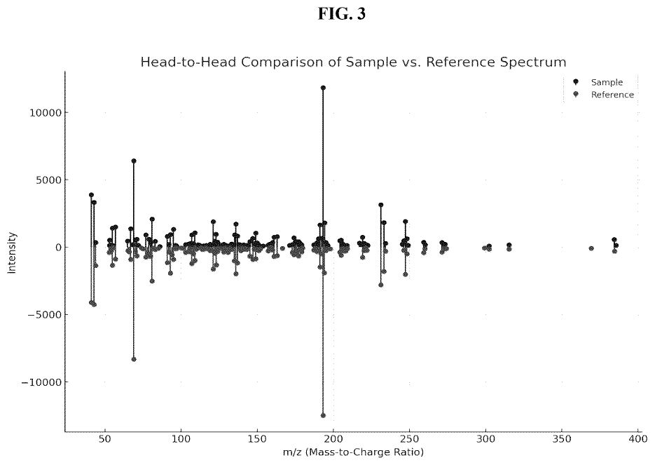

SCBG is first detected in high-THC cannabis inflorescence using Comprehensive 2-Dimensional gas chromatography coupled to time-of-flight mass spectrometry (FIG. 2). This compound elutes well beyond the cannabinoid region due to its larger size. This peak has a mass spectrum with similar mass-to-charge ratios to CBG, such as peaks located at m/z=69, 193, and 314. However, close inspection of the data reveals a parent m/z~384, which corresponds to SCBG. Mass spectra of this peak from Sample 1 is shown in FIG. 3 along with the mass spectra of the synthesized SCBG reference standard, showing excellent agreement.

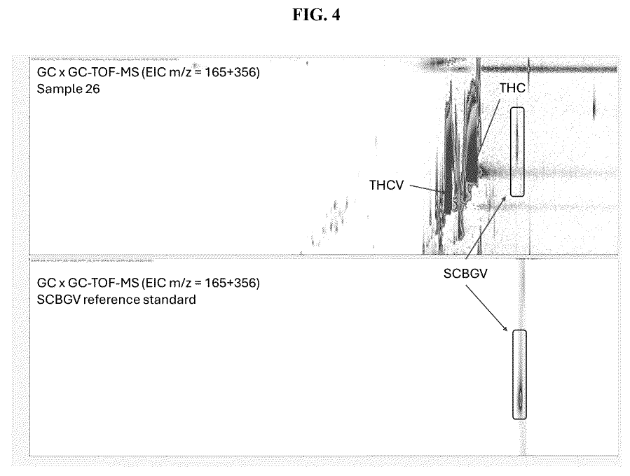

After identifying SCBG in high-THC cannabis, a high-THCV (2:1 THC: THCV) refined oil is measured. SCBGV is detected in this sample 26 (as shown in the GC×GC-TOF-MS chromatograms in FIG. 4), indicating that the varin analog is also produced by the plant, albeit in trace quantities. Similar to SCBG, the mass spectrum is very similar to CBGV (cannabigerovarin), however with a parent m/z~356, indicative of SCBGV. Mass spectra of this peak from Sample is shown in FIG. 5 which also shows the mass spectra of the SCBGV standard. Minor change in second retention time between Sample 26 and SCBGV standard is due to column degradation between experiments. Extra peaks in Sample 26 near m/z=300-320 are interference from neighboring co-eluting compound.

### Example 7: Analysis of Correlation Between SCBG and Terpene Components

The dominant volatiles and SCBG are further analyzed in total 25 different high-THC cannabis varieties or products. Samples are chosen to maximize the distribution of terpene classes as much as possible to identify potential relationships between their purported psychoactive effects and SCBG. Four types of correlation coefficients between SCBG and other components' concentrations are shown in FIG. 7, where “PEARSON,” “SPEARMAN,” “KENDALL,” and “DCOR” represent Pearson correlation coefficient (PCC), Spearman's p, Kendall's t, and distance correlation, respectively. Distance correlation is included as a nonnegative metric due to its high sensitivity. Note values distance correlation are nonnegative by definition.

For most components, the signs and relative magnitudes of PCC, p, and t agree with each other, and strong correlations can be claimed between some terpene components and SCBG. Some of these strong correlations can be intuitively identified from the regression plots. For example, strong correlations are found for (D-limonene, SCBG), as well as (β-pinene, SCBG). This supports the development of formulations comprising SCBG or SCBGV in combination with specific cannabinoids, terpenes, and other active ingredients that exhibit positive correlations with the prenylated cannabinoids and may contribute to synergistic therapeutic effects through entourage mechanisms.

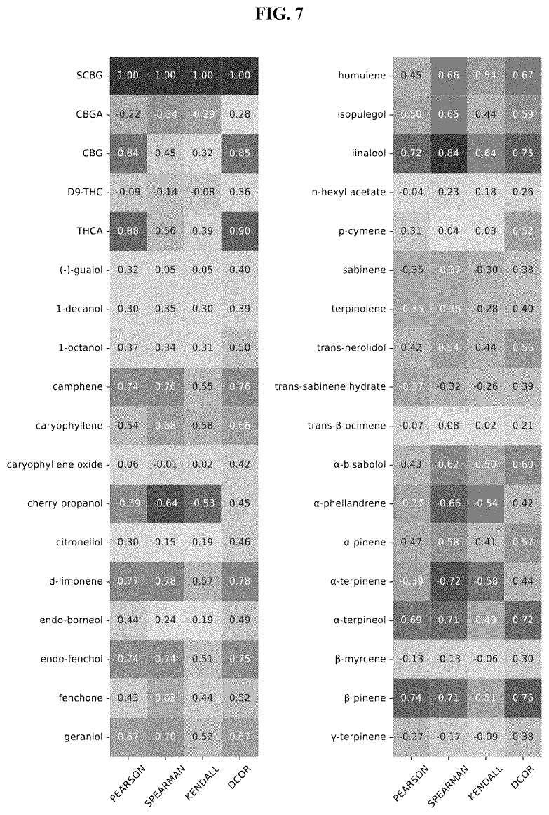

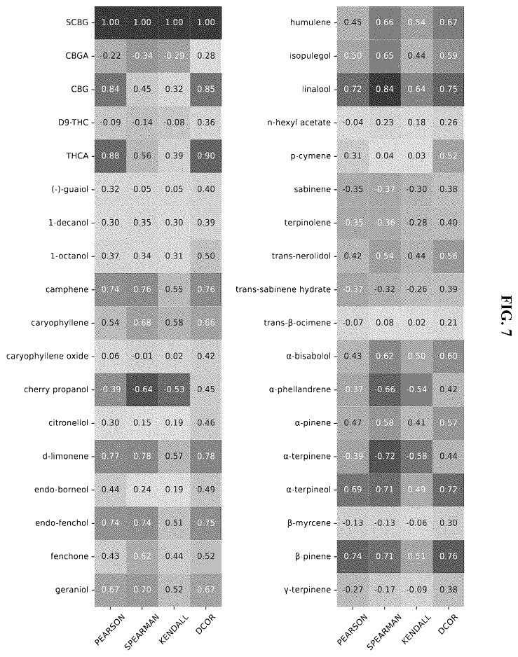

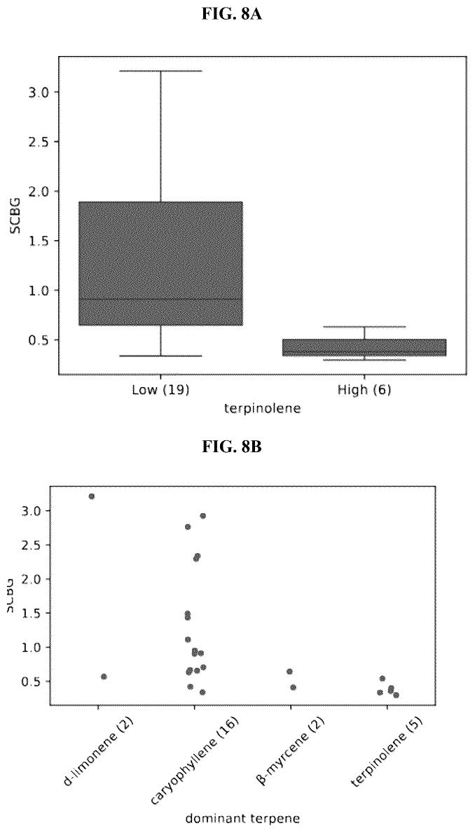

Strong and moderate negative correlations are particularly interesting as they may imply competing biosynthetic pathways. Components negatively correlated with SCBG include cherry propanol, α-phellandrene, α-terpinene, and terpinolene. Importantly, these compounds correlate to terpinolene-rich varieties, indicating that SCBG concentrations tends to be inversely related with this chemical profile. FIG. 8A shows the distinctions between low-terpinolene and high terpinolene varieties and distinct distributions of SCBG concentrations where high terpinolene samples tend to have very low SCBG concentrations, with the latter having statistically significant lower SCBG concentrations than the others. FIG. 8B shows terpinolene-rich varieties to have consistently low SCBG concentrations.

### Classification Tasks Using Logistic Regression

Two different classification tasks are explored using logistic regression:
1. Predict if a sample is a high-terpinolene sample given the concentrations of other components.
2. Predict the dominant terpene of a sample from four types of terpene given the concentrations of other components.

To avoid trivial models, data regarding the four possible dominant terpenes are removed from the model input. The input feature (component) set is determined either by an expert or through ANOVA (Analysis of Variance) feature selection. It is found that ANOVA-based feature selection usually performs better in terms of prediction performance, measured by the weighted F1 score. Model parameters and results are summarized in Table 2. Interestingly, the same feature set of {α-terpinene, α-phellandrene, γ-terpinene, cherry propanol, sabinene}, while being ordered differently, is selected for both tasks using ANOVA feature selection.

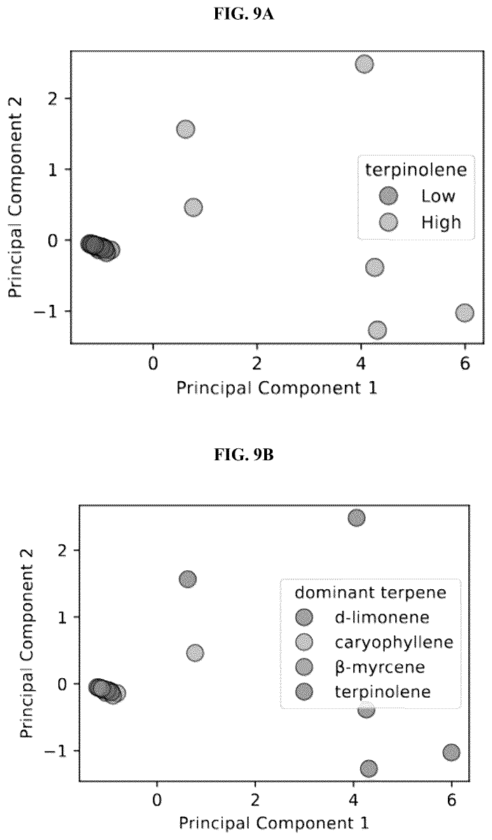

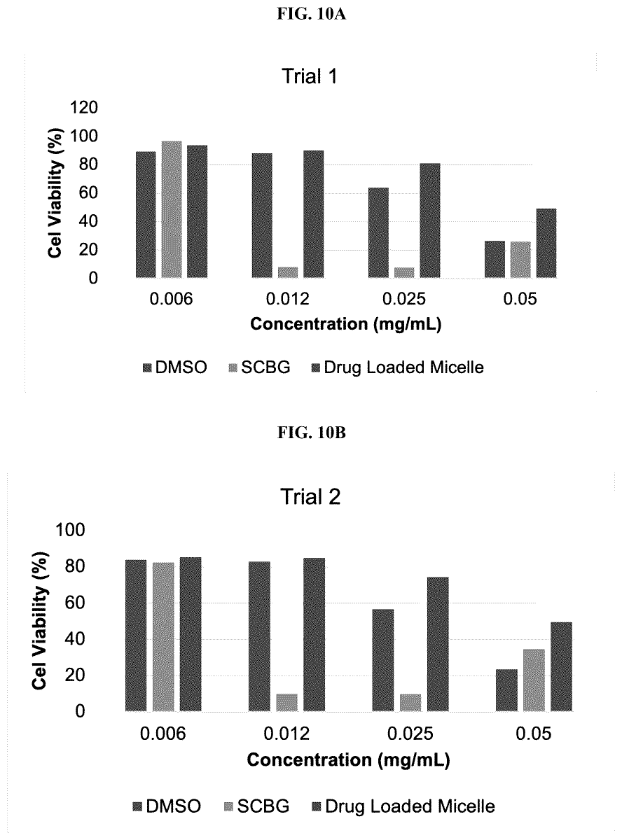

Principal Component Analysis (PCA) plots using label sets from both tasks 1 and 2 are shown in FIGS. 9A and 9B, respectively. These plots indicate that while this feature set is effective at discriminating task 1 labels and terpinolene-dominant samples, it struggles with other labels, as corroborated by the results in Table 2. Nonetheless, the results from Task 1 highlight the effectiveness of separating high-terpinolene varieties from low-terpinolene varieties further substantiates this relationship. These relationships indicate that the psychoactive effects associated with terpinolene-rich varieties (often considered “sativa”) maybe influenced by the lower SCBG concentrations. When classifying high- and low-terpinolene varieties, distinct separations between the groups are observed.

### Example 8: Molecular Docking Simulations

The molecular docking studies are performed using AutoDock Vina (J. Eberhardt, D. Santos-Martins, A. F. Tillack, and S. Forli. (2021). AutoDock Vina 1.2.0: New Docking Methods, Expanded Force Field, and Python Bindings. Journal of Chemical Information and Modeling.; O. Trott, A. J. Olson, AutoDock Vina: improving the speed and accuracy of docking with a new scoring function, efficient optimization and multithreading, Journal of Computational Chemistry 31 (2010) 455-461) via the AMDock software suite (Valdes-Tresanco, M. S., Valdes-Tresanco, M. E., Valiente, P. A. and Moreno E. AMDock: a versatile graphical tool for assisting molecular docking with Autodock Vina and Autodock4. Biol Direct 15, 12 (2020). Crystal structures of the human CB1 and CB2 receptors were obtained from the Protein Data Bank to serve as receptor models. Ligand structures of prenylated cannabinoids with alkyl side chain lengths ranging from n=1 to 10 carbons were modeled and prepared for docking simulations using Avogadro. Ligand structures are first relaxed (UFF) to minimize their energy. Tables 1 and 2 show the binding affinity of various prenylated cannabinoids as with different chemical functionalities (of alkyl carbon chains, neutral vs acidic forms).

To understand how these compounds may interact with the cannabinoid receptors, molecular docking simulations are conducted as discussed above, for each compound as shown in Table 2 and Table 3 along with their key docking results. The results revealed that all compounds exhibited strong binding affinities toward the CB2 receptor, with binding energies greater than-9 kcal/mol. Each compound has the highest affinity for the orthosteric site of the CB2 receptor. For reference, CBD docking results are shown in Table 2 as well for comparison: CBD has weaker affinity than all prenylated cannabinoid analogs, further suggesting these compounds stronger affinity over compounds traditionally associated with the cannabinoid receptors. Among the samples measured, the highest affinity is observed for compounds with side chain lengths of three to five carbons. In contrast, binding energies at the CB1 receptor are less than-7 kcal/mol, indicating low affinity for these compounds at this receptor. This suggests a reduced likelihood of psychoactive effects mediated by CB1 activation. Additionally, the acidic forms of the compounds also exhibited strong CB2 receptor affinity, maintaining binding profiles similar to their neutral counterparts.

TABLE 2
Molecular docking results showing low affinity for CB1 receptor.

| | Affinity | Affinity | Estimated | Estimated |
|---|---|---|---|---|
| # of alkyl | (−kcal/mol) | (−kcal/mol) | Ki (uM) | Ki (uM) |
| carbons | Neutral form | Acidic Form | Non-Acid | Acid Form |
| 0 | 6.5 | 7.0 | 17.2 | 7.4 |
| 1 | 6.3 | 6.9 | 24.1 | 8.76 |
| 2 | 6.7 | 6.9 | 12.27 | 8.76 |
| 3 | 6.6 | 6.9 | 14.53 | 8.76 |
| 4 | 6.3 | 7.0 | 24.1 | 7.4 |
| 5 | 6.5 | 6.4 | 17.2 | 20.36 |
| 6 | 6.6 | 6.8 | 14.53 | 10.37 |
| 7 | 6.1 | 6.6 | 33.78 | 14.53 |
| 8 | 6.3 | 6.5 | 24.1 | 17.2 |
| 9 | 6.2 | 6.3 | 28.53 | 24.1 |
| 10 | 6.4 | 6.0 | 20.36 | 39.99 |

TABLE 3
Molecular docking results showing low affinity for CB2 receptor.

| | Affinity | Affinity | Estimated | Estimated |
|---|---|---|---|---|
| # of alkyl | (−kcal/mol) | (−kcal/mol) | Ki (nM) | Ki (nM) |
| carbons | Non-Acid | Acid Form | Non-Acid | Acid Form |
| 0 | 9.1 | 9.3 | 213.63 | 152.43 |
| 1 | 9.2 | 9.2 | 180.45 | 180.45 |
| 2 | 9.4 | 9.7 | 128.75 | 77.60 |
| 3 | 9.7 | 9.8 | 77.60 | 65.55 |
| 4 | 9.5 | 10.1 | 108.76 | 39.51 |
| 5 | 9.5 | 9.5 | 108.76 | 108.76 |
| 6 | 9.6 | 9.6 | 91.87 | 91.87 |
| 7 | 9.6 | 9.7 | 91.87 | 77.60 |
| 8 | 9.6 | 9.8 | 91.87 | 65.55 |
| 9 | 9.6 | 9.7 | 91.87 | 77.60 |
| 10 | 9.7 | 9.7 | 77.60 | 77.60 |
| CBD | 8.9 | NA | 299.41 | NA |

The structures of illustrative compounds of the present technology having varying length of alkyl carbons are shown in FIG. 1A and Table 1. The chemical reactivity of the C—H groups on the aromatic ring in these compounds suggests that further functionalization is possible and may have further enhanced or modified effects. For instance, SCBGPr, (R=farnesyl, R₁=prenyl), has a high docking affinity of −10.4 kcal/mol and 23.81 nm inhibition constant. This provides good evidence that further modification of these compounds may provide further utility.

### Example 9: Compositions of Prenylated Cannabinoids

Various forms of compositions containing the of prenylated cannabinoids were prepared. Table 4 shows illustrative products containing SCBG with other ingredients.

TABLE 4
Examples of prenylated cannabinoid formulation compositions.

| Ex. | Product | SCBG | | Other | | | | |
|---|---|---|---|---|---|---|---|---|
| No. | Type | Content | | Cannabinoids | | Carrier | Other Ingredients | Application |
| 1 | Oil tincture | 10 | mg | 200 | mg CBD | MCT | Myrcene, limonene | Sublingual/ |
| | | | | | | Oil | (flavors and | oral |
| | | | | | | | terpenes) | |
| 2 | Edible | 5 | mg | 2 mg THC, 2 | | Gelatin | Citric acid (flavor), | Oral/edible |
| | gummy | | | mg CBD | | base | ascorbic acid | |
| | | | | | | | (antioxidant) | |
| 3 | Beverage | 0.5 | mg | None | | Water | Lemon flavor, | Oral/beverage |
| | (12 oz | | | | | | Stevia (sweetener), | |
| | drink) | | | | | | potassium sorbate, | |
| | | | | | | | lecithin | |
| 4 | Topical | 1 | g | None | | Shea | Lavender oil, | Topical |
| | cream | | | | | butter, | eucalyptus oil | application |
| | | | | | | jojoba | (aromatics) | |
| | | | | | | oil | | |
| 5 | Vape liquid | 50 | mg | None | | Propylene | Limonene, beta- | Inhalable |
| | | | | | | glycol | caryophyllene | |
| | | | | | | | (terpenes) | |
| 6 | Capsule | 25 | mg | 5 | mg CBG | Olive oil | Gelatin (capsule | Oral/ |
| | | | | | | | shell), Vitamin E | nutraceutical |
| | | | | | | | (antioxidant) | |
| 7 | Tablet | 10 | mg | None | | Microcrystalline | Magnesium stearate | Oral/ |
| | | | | | | cellulose | (binder), peppermint | nutraceutical |
| | | | | | | | flavoring | |
| 8 | Transdermal | 100 | mg | None | | Silicone-based | Capsaicin | Transdermal |
| | patch | | | | | matrix | (permeation | |
| | | | | | | | enhancer), Glycerin | |
| 9 | Oral spray | 15 | mg | None | | Ethanol | Mint flavor, | Oral/ |
| | (sublingual) | | | | | | glycerin | sublingual |
| | | | | | | | (sweetener) | |

| | | | | | | | |
|---|---|---|---|---|---|---|---|
| 10 | Protein bar | 2 | mg | None | None | Peanut butter, | Oral/edible |

honey, oats, soy
protein

### Example 10: Drug-Loaded Micelles (DLM) Preparation

Stock DLM includes 0.1 mg SCBG+1 mg Polymer (2000mwPEG-5000mwPCL) in 1 mL water. 100 μL of cell culture media is used to grow cells in each well (96-well plate). The serial dilution is prepared in culture media, which is equal across all trials to limit variables. 500 μL of stock DLM+500 μL culture media is mixed and 100 μL of the solution is then dosed into a well to create 0.05 mg/mL. 500 μL of the 0.05 mg/mL solution+500 μL culture media is mixed and 100 μL of the solution is then dosed into a well to create 0.025 mg/mL concentration. Further, 500 L of the 0.025 mg/mL solution+500 μL culture media is mixed and 100 μL of the solution is then dosed into a well to create 0.012 mg/mL concentration. Finally, 500 μL of the 0.012 mg/mL solution+500 μL culture media is mixed and 100 μL of the solution is then dosed into a well to create 0.006 mg/mL concentration.

### Example 11: SCBG Cell Culture Preparation and Analysis

Stock solution includes 1 mg SCBG in 1 mL DMSO. 50 mL stock+950 μL cell culture media, then dose 100 μL of this into well. Creates 0.050 mg/mL concentration.

50 μL of stock+950 μL culture media is mixed and 100 μL of the solution is then dosed into a well to create 0.05 mg/mL. 25 μL of the 0.05 mg/mL solution+975 μL culture media is mixed and 100 μL of the solution is then dosed into a well to create 0.025 mg/ml concentration. Further, 12 μL of the 0.05 mg/mL solution+988 μL culture media is mixed and 100 μL of the solution is then dosed into a well to create 0.012 mg/mL concentration. Finally, 6 μL of the 0.05 mg/mL solution+994 μL culture media is mixed and 100 μL of the solution is then dosed into a well to create 0.006 mg/mL concentration.

### Example 12: DMSO as a Control Preparation

The following solutions were prepared as controls:
50 μL DMSO+950 μL cell culture media (control for 0.05 mg/mL dmso-scbg)
25 μL DMSO+975 μL cell culture media (control for 0.025 mg/mL dmso-scbg)
12 μL DMSO+988 μL cell culture media (control for 0.012 mg/mL dmso-scbg)
6 μL DMSO+994 μL cell culture media (control for 0.006 mg/mL dmso-scbg)

The trial composition data is shown in Table 5 below.

TABLE 5

| | | |
|---|---|---|
| Trial | Concentration | Carrier |

| | | | |
|---|---|---|---|
| SCBG in DMSO | 0.05 | mg/mL | DMSO |
| SCBG in DMSO | 0.025 | mg/mL | DMSO |
| SCBG in DMSO | 0.012 | mg/mL | DMSO |
| SCBG in DMSO | 0.006 | mg/mL | DMSO |
| DMSO | 0.05 | mg/mL | DMSO |
| DMSO | 0.025 | mg/mL | DMSO |
| DMSO | 0.012 | mg/mL | DMSO |
| DMSO | 0.006 | mg/ml | DMSO |
| SCBG drug loaded micelle | 0.05 | mg/mL | culture media |
| SCBG drug loaded micelle | 0.025 | mg/mL | culture media |
| SCBG drug loaded micelle | 0.012 | mg/mL | culture media |
| SCBG drug loaded micelle | 0.006 | mg/mL | culture media |

### Example 13: Cell Viability Test Method

MDA-MB-231 cells are plated on a 96-well plate at a density of 10,000 cells per well and cultured in 100 mL growth media. Before adding any of the trials, the old growth media is removed and replaced with 120 mL of fresh growth media. All drugs (drug loaded micelles, drug diluted in DMSO, and control DMSO) are added as final dose (mg indicated) into cells. DMSO is used as a control for the drug diluted in DMSO, for the drug dosages see Table 5. The cells are incubated at 37° C. for 4 hours, then washed in PBS, and then incubated for another 24 hours. After fixation (4% PFA for 15 minutes at room temperature followed by washing twice with PBS for 5 minutes PBS and DAPI staining, 300 nM DAPI in PBS, for 15 minutes at room temperature followed by washing twice with PBS for 5 minutes) the cells are imaged on a Biotech Cytation3 Cell Imaging multimode Reader.

FIGS. 11A and 11B-Trial 1 and Trial 2 demonstrates that the SCBG has significant cancer cell treatment viability when diluted in a carrier solvent and specifically more effective than the drug loaded micelles. The drug loaded micelles have the advantage of delivering the drug (SCBG) directly inside the cell and typically to the nucleus of the cell. However, this data suggests that the mechanism for action is not like other cancer cell drugs that operate on the principle of disrupting the nucleus and DNA of the cell but rather another mechanism. This unorthodox approach of reducing proliferation is attractive for a few reasons. First, it avoids complex encapsulation technologies that are typically required for anticancer drugs. Second, the steep drop in cell viability between the lowest concentration (6 μg/mL; ~100% viability) and the second lowest (12 μg/mL; ~ 5% viability) indicates that the cells are most likely being killed via a mechanistic pathway wherein the SCBG interacts with the surface to induce apoptosis. This may be due to the compound interacting with the membrane, CB2 activation (SCBG has strong affinity for CB2), or a combination of receptors.

Several cannabinoids are converted into other metabolites in the human body, often through the liver. These metabolites frequently possess unique and useful chemical properties as well. As such, it is hypothesized that the metabolites produced by metabolism of prenylated cannabinoids likewise may possess similar properties to their typical phytocannabinoids such as THC, CBD, or CBG. The production of these metabolites may further enhance the efficacy of the original substance.

### Example 14. Receptor Binding and Functional Activity Profile

In certain embodiments, the compounds disclosed herein were evaluated for activity at cannabinoid receptors CB1 and CB2, as well as selected transient receptor potential (TRP) channels, including TRPV1, TRPA1, and TRPM8. The compounds comprise a homologous series of prenylated cannabinoid analogs, including but not limited to SCBGV, SCBGB, SCBG, SCBGP, and SCBGG.

TRPM8 assay. The high-throughput screening (HTS) to identify TRPM8 inhibitors was conducted by Eurofins Discovery following previously established protocols (Br. J. Pharmacol., 141:737-745, 2004). This was a functional cell based fluorometric assay with BCTC used as an antagonist control and icilin as the agonist control.

TRPV1 assay. The high-throughput screening (HTS) to identify TRPV1 activators was conducted by Eurofins Discovery following previously established protocols (Eur. J. Pharmacol., 513:57-66, 2005). This was a functional cell based fluorometric assay with Capsaicin used as the activator control with olvanil and AM404 as positive controls.

TRPA1 assay. The high-throughput screening (HTS) to identify TRPA1 activators was conducted by Eurofins Discovery following previously established protocols (Physiol. Rev. April 1; 100 (2): 725-803, 2020). This was a functional cell based fluorometric assay developed in HEK293 cells with J1010 used as the activator control with allyl isothiocyanate as positive control.

CB1 Human cannabinoid GPCR binding assay. The biochemical assay screening for CB1 binding was conducted by Eurofins Discovery following previously established protocols (J. Pharmacol. Exp. Ther., 278:871-878, 1996). This was a binding assay using radiometric detection with [H3] CP55940 as the ligand at 2 nM, CP55940 and WIN 55212-2 as the control inhibitors, and AM281 (10 μM) as the nonspecific in the assay.

CB2 Human cannabinoid GPCR binding assay. The biochemical assay screening for CB2 binding was conducted by Eurofins Discovery following previously established protocols (Nature, 365:61-65, 1993). This is a binding assay using radiometric detection with [H3] WIN 55212-2 as the ligand, and WIN 55212-2 as the control inhibitor, and as the nonspecific control (5 μM) in the assay. Results. CB1 and CB2 receptor binding was assessed at a test concentration of 10 μM.

Notably, compounds such as SCBGV and SCBGB exhibited strong CB2 binding with minimal or absent CB1 binding, indicating a preferential CB2 receptor affinity. Other compounds in the series showed weak CB1 interaction while retaining low-to-moderate CB2 activity. This receptor selectivity profile is advantageous for applications where peripheral cannabinoid signaling is desired without central CB1-mediated psychoactivity.

In addition to cannabinoid receptor binding, the compounds were evaluated for functional activity at TRPV1, TRPA1, and TRPM8 channels. Across the homologous series, the compounds demonstrated consistent activity at one or more TRP channels, with particularly strong activity observed at TRPM8 in multiple analogs. As summarized in Table 6 below, the compounds consistently exhibited minimal to negligible binding at the CB1 receptor, while several members of the series demonstrated measurable to pronounced binding at the CB2 receptor.

TABLE 6
Summary of receptor binding assay
for select prenylated cannabinoids.

| | CB1 | CB2 | TRPV1 | TRPA1 | TRPM8 |
|---|---|---|---|---|---|
| Compound | Binding* | Binding** | EC₅₀ | EC₅₀ | EC₅₀ |
| SCBGV | + | +++ | + | ++ | +++ |
| SCBGB | − | +++ | + | ++ | +++ |
| SCBG | + | + | + | ++ | +++ |
| SCBGP | + | ++ | −/+ | ++ | ++++ |
| SCBGG | − | − | −/+ | + | ++++ |

*CB1 binding % at 10 uM,
**CB2 binding % at 10 Um.

Unless otherwise expressly stated, it is in no way intended that any method set forth herein be construed as requiring that its steps be performed in a specific order, nor that with any apparatus specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred, in any respect. This holds for any possible non-express basis for interpretation, including: matters of logic with respect to arrangement of steps, operational flow, order of components, or orientation of components; plain meaning derived from grammatical organization or punctuation, and; the number or type of embodiments described in the specification.

All disclosed ranges are to be understood to encompass and provide support for claims that recite any and all subranges or any and all individual values subsumed by each range. For example, a stated range of 1 to 10 should be considered to include and provide support for claims that recite any and all subranges or individual values that are between and/or inclusive of the minimum value of 1 and the maximum value of 10; that is, all subranges beginning with a minimum value of 1 or more and ending with a maximum value of 10 or less (e.g., 5.5 to 10, 2.34 to 3.56, and so forth) or any values from 1 to 10 (e.g., 3, 5.8, 9.9994, and so forth). Any listed range may be easily recognized as sufficiently describing and enabling the same range being broken down into at least equal halves, thirds, quarters, fifths, tenths, etc. As a non-limiting example, each range discussed herein may be readily broken down into a lower third, middle third and upper third, etc. As will also be understood by one skilled in the art all language such as “up to,” “at least,” “greater than,” “less than,” and the like, include the number recited and refer to ranges which may be subsequently broken down into subranges as discussed above. Finally, as will be understood by one skilled in the art, a range includes each individual member. Thus, for example, a group having 1-3 layers refers to groups having 1, 2, or 3 layers. Similarly, a group having 1-5 layers refers to groups having 1, 2, 3, 4, or 5 layers, and so forth.

The drawings shall be interpreted as illustrating one or more embodiments that are drawn to scale and/or one or more embodiments that are not drawn to scale. This means the drawings may be interpreted, for example, as showing: (a) everything drawn to scale, (b) nothing drawn to scale, or (c) one or more features drawn to scale and one or more features not drawn to scale. Accordingly, the drawings can serve to provide support to recite the sizes, proportions, and/or other dimensions of any of the illustrated features either alone or relative to each other. Furthermore, all such sizes, proportions, and/or other dimensions are to be understood as being variable from 0-100% in either direction and thus provide support for claims that recite such values or any and all ranges or subranges that may be formed by such values.

The terms recited in the claims should be given their ordinary and customary meaning as determined by reference to relevant entries in widely used general dictionaries and/or relevant technical dictionaries, commonly understood meanings by those in the art, etc., with the understanding that the broadest meaning imparted by any one or combination of these sources should be given to the claim terms (e.g., two or more relevant dictionary entries should be combined to provide the broadest meaning of the combination of entries, etc.) subject only to the following exceptions: (a) if a term is used in a manner that is more expansive than its ordinary and customary meaning, the term should be given its ordinary and customary meaning plus the additional expansive meaning, or (b) if a term has been explicitly defined to have a different meaning by reciting the term followed by the phrase “as used in this document shall mean” or similar language (e.g., “this term means,” “this term is defined as,” “for the purposes of this disclosure this term shall mean,” etc.). References to specific examples, use of “i.e.,” use of the word “technology,” etc., are not meant to invoke exception (b) or otherwise restrict the scope of the recited claim terms. Other than situations where exception (b) applies, nothing contained in this document should be considered a disclaimer or disavowal of claim scope.

Unless otherwise defined, all terms (including technical and scientific terms) used herein have the same meaning as commonly understood by one of ordinary skill in the art to which this technology belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

In addition, where features or aspects of the disclosure are described in terms of Markush groups, those skilled in the art will recognize that the disclosure is also thereby described in terms of any individual member or subgroup of members of the Markush group.

Unless the context indicates otherwise, it is specifically intended that the various features of the technology described herein may be used in any combination. Moreover, the disclosure also contemplates that in certain embodiments, any feature or combination of features set forth herein may be excluded or omitted. To illustrate, if the specification states that a complex comprises components A, B and C, it is specifically intended that any of A, B or C, or a combination of any two or more thereof, may be omitted and disclaimed singularly or in any combination.

Unless explicitly indicated otherwise, all specified embodiments, features, and terms intend to include both the recited embodiment, feature, or term and biological equivalents thereof.

All patents, patent applications, provisional applications, and publications referred to or cited herein are incorporated by reference in their entirety, including all figures and tables, to the extent they are not inconsistent with the explicit teachings of this specification.

Other embodiments are set forth in the following claims. cm What is claimed is:

1. A composition comprising:
a prenylated cannabinoid in an amount effective to modulate cannabinoid receptor CB2 activity; and
a pharmaceutically or nutraceutically acceptable carrier;
wherein:
the prenylated cannabinoid has the following formula (I): 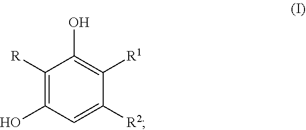
R at each occurrence is farnesyl or geranylgeranyl;
R¹ at each occurrence is hydrogen or —COOR³;
R² at each occurrence is-C₃-C₂₀ alkyl; and
R³ at each occurrence is selected from hydrogen, —C₁-C₂₀ alkyl, and —C₁-C₂₀ haloalkyl.

2. The composition of claim 1, wherein the prenylated cannabinoid comprises sesqui-cannabigerol (SCBG), sesqui-cannabigerovarin (SCBGV), sesqui-cannabigerobutyl (SCBGB), sesqui-cannabigerophorol (SCBGP) sesqui-cannabigeranylgerol (SCBGG), acidic derivatives thereof, alkyl ester derivatives thereof, or a combination of any two or more thereof.

3. The composition of claim 1, wherein the prenylated cannabinoid comprises sesqui-cannabigeranylgerol (SCBGG) having the following formula: 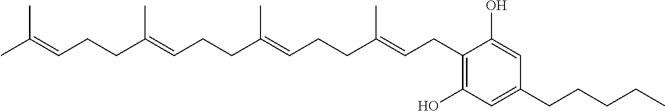 [SMILES: CCCCCc1cc(O)c(C/C=C(\C)CC/C=C(\C)CC/C=C(\C)CCC=C(C)C)c(O)c1]
or a pharmaceutically acceptable salt, ester, or acidic derivative thereof.

4. The composition of claim 1, comprising at least 0.001% by weight of the one or more prenylated cannabinoids by weight of the total composition.

5. The composition of claim 1, wherein the composition comprises between 0.1 mg and 500 mg of the one or more prenylated cannabinoids per unit dose.

6. The composition of claim 1, wherein the one or more prenylated cannabinoids is present at a concentration higher than its natural abundance in cannabis plants.

7. The composition of claim 1, wherein the prenylated cannabinoid exhibits higher or preferential binding affinity for CB2 relative to CB1.

8. The composition of claim 1, wherein the carrier comprises an oil, solvent, emulsifier, or polymer.

9. The composition of claim 1, further comprising an additional cannabinoid comprising cannabidiol, cannabigerol, cannabinol, tetrahydrocannabinol, tetrahydrocannabivarin, acidic forms thereof, and combinations of any two or more thereof.

10. The composition of claim 1 further comprising, a terpene and/or a flavorant.

11. The composition of claim 1, wherein the composition is a beverage, alcoholic beverage, herbal supplement, nutraceutical supplement, protein supplement, pre-workout supplement, ready to mix beverage powder, brewed beverage, fermented beverage, edible product, candy, baked good, food product, tincture, capsule, tablet, cream, lotion, or vape liquid.

12. The composition of claim 1, wherein R is geranylgeranyl.

13. Sesqui-cannabigeranylgerol (SCBGG) or a pharmaceutically acceptable salt, ester, or acidic derivative thereof.

14. A composition comprising the compound of claim 13 and a pharmaceutically or nutraceutically acceptable carrier.

15. A method of modulating CB2 receptor activity in a subject in need thereof, comprising administering to the subject a therapeutically effective amount of the composition of claim 1.

16. The method of claim 15, wherein modulation of CB2 receptor activity results in a reduction of anxiety, paranoia, inflammation, pain, or a combination of any two or more thereof.

17. The method of claim 15, wherein the subject is experiencing anxiety or paranoia associated with administration of tetrahydrocannabinol (THC).

18. A method of treating cancer in a subject in need thereof, comprising administering an effective amount of the composition of claim 1.

19. The method of claim 18, wherein the cancer is triple-negative breast cancer.
